(19) United States
(12) Patent Application Publication
(10) Pub. No.: US 2023/0220114 A1
(43) Pub. Date: Jul. 13, 2023

(54) PURIFICATION OF MULTISPECIFIC ANTIBODIES

(71) Applicant: GENENTECH, INC., South San Francisco, CA (US)

(72) Inventors: Ambrose J. Williams, South San Francisco, CA (US); Ankai Xu, South San Francisco, CA (US)

(73) Assignee: GENENTECH, INC., South San Francisco, CA (US)

(21) Appl. No.: 18/124,096

(22) Filed: Mar. 21, 2023

Related U.S. Application Data
(63) Continuation of application No. PCT/US2021/051047, filed on Sep. 20, 2021.
(60) Provisional application No. 63/080,950, filed on Sep. 21, 2020.

(51) Int. Cl. C07K 16/46; C07K 1/16

(52) U.S. Cl. CPC C07K 16/468; C07K 1/165; C07K 2317/31; C07K 2317/66

(57) ABSTRACT

The present disclosure provides methods for purifying multispecific antibodies from a mispaired variant thereof by performing a multi-mode chromatography.

### CROSS-REFERENCE TO RELATED APPLICATIONS

This application is a continuation application of International Patent Application No. PCT/US21/51047, filed Sep. 20, 2021, which claims priority to U.S. Provisional Patent Application Ser. No. 63/080,950, filed Sep. 21, 2020, the contents of each of which are incorporated by reference in their entirety, and to each of which priority is claimed.

### FIELD

Methods for purifying multispecific antibodies from a composition comprising the multispecific antibody and at least one impurity, including at least one product-specific impurity, are provided. In some embodiments, the product-specific impurity is, for example, a mispaired variant of the multispecific antibody. Also provided are multispecific antibodies purified according to the methods, and compositions and formulations comprising such multispecific antibodies.

### BACKGROUND

For recombinant biopharmaceutical proteins to be acceptable for administration to human patients, it is important that residual impurities resulting from the manufacture and purification process are removed from the final biological product. These process components include culture medium proteins, immunoglobulin affinity ligands, viruses, endotoxin, DNA, and host cell proteins (HCPs). The development of new antibody formats, such as multispecific antibodies, presents new challenges as conventional manufacturing and purification processes are inadequate to sufficiently remove product-specific impurities, including non-paired antibody arms and misassembled antibodies.

As compared to the purification of standard antibodies, the purification of multispecific antibodies from production media presents unique challenges. While a standard mono-specific bivalent antibody results from the dimerization of identical heavy-chain/light-chain subunits, the production of a multispecific antibody requires dimerization of at least two different heavy-chain/light-chain subunits, each comprising a different heavy chain as well as a different light chain. The production and purification of the final correct and complete multispecific antibody, with minimal amounts of mis-paired, mis-assembled, or incomplete molecules present different challenges. Chain mispairings (e.g., homo-dimerization of identical heavy chain peptides or improper heavy-chain/light-chain associations) are often observed. Commonly observed product-specific impurities include half (½) antibodies (comprising a single heavy-chain/light-chain pair), three-quarter (¾) antibodies (comprising a complete antibody lacking a single light chain), and homodimers. Additional product-specific impurities may be observed depending on the multispecific format used. For example, where one variable domain of the multispecific antibody is constructed as a single-chain Fab (scFab), a 5/4 antibody by-product (comprising an additional heavy or light chain variable domain) may be observed. Such corresponding product-specific impurities would not arise in standard antibody production.

Conventional purification techniques designed to remove process-related impurities such as HCPs, DNA, endotoxins, and other materials that have very different characteristics and properties from the antibodies can be inadequate when implemented to remove impurities that are more similar to the multispecific antibodies. As such, there is a need to develop manufacturing and purification schemes that effectively remove product-specific impurities and light chain mispaired antibodies and yield sufficient amount of the correct and complete multispecific antibody.

All references cited herein, including patent applications and publications, are incorporated by reference in their entirety for any purpose.

### SUMMARY

The present disclosure provides a method for purifying a multispecific antibody, comprising: (a) contacting a composition comprising the multispecific antibody and a mispaired variant thereof to a multi-mode chromatography material under conditions where the mispaired variant preferentially binds the multi-mode chromatographic material relative to the multispecific antibody, wherein the multispecific antibody comprises a first antigen binding region specifically binding to a first antigen, wherein the first antigen binding region comprises the light chain and heavy chain of an antibody binding to the first antigen, and a second antigen binding region specifically binding to a second antigen, wherein the second antigen binding region comprises the light chain and heavy chain of an antibody binding to the second antigen, wherein in the second antigen binding region the variable domains VL and VH are replaced by each other; wherein the mispaired variant thereof comprises a first antigen binding region comprising the heavy chain of the antibody binding to the first antigen and a peptide comprising the heavy chain variable domain (VH) and the light chain constant domain (CL) of the antibody binding to the second antigen, and a second antigen binding region comprising the light chain and heavy chain of an antibody binding to the second antigen, wherein in the second antigen binding region the variable domains VL and VH are replaced by each other; and wherein the multi-mode chromatography material comprises a functional group capable of anion exchange and a functional group capable of hydrophobic interactions; and (b) collecting an eluate comprising the multispecific antibody and reduced amount of the mispaired variant thereof.

In certain embodiments, the functional group capable of hydrophobic interactions comprises an alkyl-group, an alkenyl-group, an alkynyl-group, a phenyl-group, a benzyl-group, or any combination thereof. In certain embodiments, the functional group comprises a benzyl-group. In certain embodiments, the functional group capable of anion exchange comprises a positively charged group. In certain embodiments, the positively charged group is a quaternary ammonium ion. In certain embodiments, the multi-mode chromatography material comprises a N-benzyl-N-methyl ethanolamine. In certain embodiments, the multi-mode chromatography material comprises a Capto™ Adhere resin. In certain embodiments, the multi-mode chromatography material comprises a Capto™ Adhere ImpRes resin.

In certain embodiments, the elution of the multi-mode chromatography is a gradient elution. In certain embodiments, the gradient elution comprises a pH gradient.

In certain embodiments, the method comprises a capture chromatography step. In certain embodiments, the capture chromatography step is an affinity chromatography step. In certain embodiments, the affinity chromatography step is a protein A chromatography step, a protein L chromatography step, a protein G chromatography step, and a protein A/G chromatography step. In certain embodiments, the affinity chromatography step is a protein A chromatography step. In certain embodiments, the protein A chromatography material comprises protein A linked to agarose.

In certain embodiments, the capture chromatography step and the multi-mode chromatography step are contiguous. In certain embodiments, the method further comprises a purification step after the multi-mode chromatography. In certain embodiments, the method comprises a concentration of the multispecific antibody.

In certain embodiments, the multispecific antibody comprises a knob-in-hole modification.

In certain embodiments, the multispecific antibody and the mispaired variant thereof are produced in the same host cell. In certain embodiments, the host cell is a prokaryotic cells or a eukaryotic cell. In certain embodiments, the host cells is a eukaryotic cell. In certain embodiments, the eukaryotic cell is a yeast cell, an insect cell, or a mammalian cell. In certain embodiments, the eukaryotic cell is a CHO cell.

The present disclosure provides a composition comprising a multispecific antibody purified by the method of a method disclosed herein. In certain embodiments, the composition further comprises a pharmaceutically acceptable carrier.

The present disclosure provides an article of manufacture comprising a multispecific antibody purified by a method disclosed herein. The present disclosure also provides an article of manufacture comprising a composition disclosed herein.

### BRIEF DESCRIPTION OF THE DRAWINGS

FIGS. 1A-1B depict a schematic overview of the method for producing multispecific antibodies. FIG. 1A shows an overview of the production of multispecific antibodies by using a two-cell approach. FIG. 1B shows an overview of the production of multispecific antibodies by using a single-cell approach.

FIGS. 2A-2B depict a representation of variants of multispecific antibodies. FIG. 2A shows a schematic table of the different covalent dimers and light-chain mispair variants. FIG. 2B shows a representation of a correctly formed bispecific antibody (left panel) and of a crossed light-chain mispair variant (right panel).

FIG. 3 shows contour plots depicting strong binding of common crossed LC mispair variant to resin, under conditions where binding of bispecific is minimal.

FIG. 4 shows a chromatogram displaying pH, UV Absorbance, elution mixture gradient, and conductivity.

FIG. 5 shows mass spectrometry data comparing load feedstock composition to fractions representing multispecific antibodies and LC-mispair variants.

FIGS. 6A-6B show pseudo-chromatograms depicting composition and concentration of collected and measured fractions. FIG. 6A shows that the main peak comprises primarily bispecific antibodies. FIG. 6B shows the normalized pseudo-chromatograms for bispecific and LC-mispair variants.

FIG. 7 shows in silico structural analysis of correct paired multispecific antibodies and LC-mispaired variants.

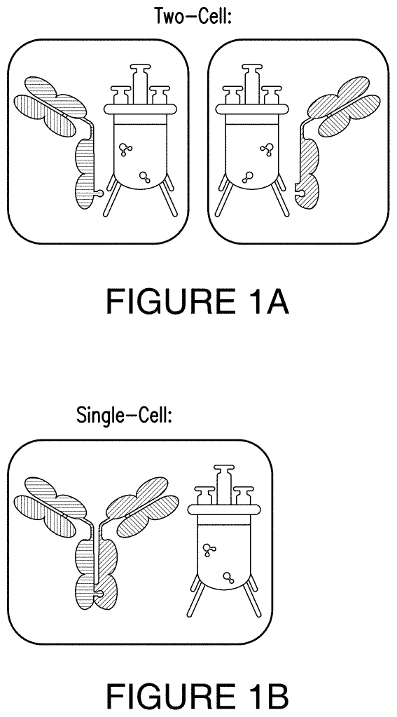

### DETAILED DESCRIPTION

The present disclosure is based, at least in part, on the finding that it is possible to remove mispaired variants of multispecific antibodies produced by a same cell by performing a multi-mode chromatography. The present disclosure surprisingly shows that a multi-mode chromatography is able to separate desired multispecific CrossMab antibody from unwanted variants thereof.

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

As used in this specification and the appended claims, the singular forms “a,” “an” and “the” include plural referents unless the context clearly dictates otherwise. Thus, for example, reference to “a protein” or an “antibody” includes a plurality of proteins or antibodies, respectively; reference to “a cell” includes mixtures of cells, and the like.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Reference to “about” a value or parameter herein includes (and describes) embodiments that are directed to that value or parameter per se. For example, description referring to “about X” includes description of “X.”

The terms “polypeptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. The terms “polypeptide” and “protein” as used herein specifically encompass antibodies.

“Purified” polypeptide (e.g., antibody or immunoadhesin) means that the polypeptide has been increased in purity, such that it exists in a form that is more pure than it exists in its natural environment and/or when initially synthesized and/or amplified under laboratory conditions. Purity is a relative term and does not necessarily mean absolute purity. The terms “purifying,” “separating,” or “isolating,” as used interchangeably herein, refer to increasing the degree of purity of a desired molecule (such as a multispecific antibody, e.g., a bispecific antibody) from a composition or sample comprising the desired molecule and one or more impurities. Typically, the degree of purity of the desired molecule is increased by removing (completely or partially) at least one impurity from the composition.

A multispecific antibody “which binds an antigen of interest” is one that binds the antigen, e.g., a protein, with sufficient affinity such that the multispecific antibody is useful as a diagnostic and/or therapeutic agent in targeting a protein or a cell or tissue expressing the protein, and does not significantly cross-react with other proteins. In such embodiments, the extent of binding of the multispecific antibody to a “non-target” protein will be less than about 10% of the binding of the multispecific antibody to its particular target protein as determined by, e.g., fluorescence activated cell sorting (FACS) analysis, radioimmunoprecipitation (RIA), or ELISA, etc. With regard to the binding of a multispecific antibody to a target molecule, the term “specific binding” or “specifically binds to” or is “specific for” a particular polypeptide or an epitope on a particular polypeptide target means binding that is measurably different from a nonspecific interaction (e.g., a non-specific interaction may be binding to bovine serum albumin or casein). Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of at least about 200 nM, alternatively at least about 150 nM, alternatively at least about 100 nM, alternatively at least about 60 nM, alternatively at least about 50 nM, alternatively at least about 40 nM, alternatively at least about 30 nM, alternatively at least about 20 nM, alternatively at least about 10 nM, alternatively at least about 8 nM, alternatively at least about 6 nM, alternatively at least about 4 nM, alternatively at least about 2 nM, alternatively at least about 1 nM, or greater affinity. In one embodiment, the term “specific binding” refers to binding where a multispecific antigen-binding protein binds to a particular polypeptide or epitope on a particular polypeptide without substantially binding to any other polypeptide or polypeptide epitope.

“Binding affinity” generally refers to the strength of the sum total of noncovalent interactions between a single binding site of a molecule (e.g., a multispecific antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). For example, the Kd can be about 200 nM or less, about 150 nM or less, about 100 nM or less, about 60 nM or less, about 50 nM or less, about 40 nM or less, about 30 nM or less, about 20 nM or less, about 10 nM or less, about 8 nM or less, about 6 nM or less, about 4 nM or less, about 2 nM or less, or about 1 nM or less. Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the methods and compositions provided herein.

“Active” or “activity” for the purposes herein refers to form(s) of a polypeptide (such as a multispecific antibody) which retain a biological and/or an immunological activity of native or naturally-occurring polypeptide, wherein “biological” activity refers to a biological function (either inhibitory or stimulatory) caused by a native or naturally-occurring polypeptide other than the ability to induce the production of an antibody against an antigenic epitope possessed by a native or naturally-occurring polypeptide and an “immunological” activity refers to the ability to induce the production of an antibody against an antigenic epitope possessed by a native or naturally-occurring polypeptide.

“Biologically active” and “biological activity” and “biological characteristics” with respect to a multispecific antigen-binding protein provided herein, such as an antibody, fragment, or derivative thereof, means having the ability to bind to a biological molecule, except where specified otherwise.

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term “immunoglobulin” (Ig) is used interchangeable with antibody herein.

Antibodies are naturally occurring immunoglobulin molecules which have varying structures, all based upon the immunoglobulin fold. For example, IgG antibodies have two “heavy” chains and two “light” chains that are disulfide-bonded to form a functional antibody. Each heavy and light chain itself comprises a “constant” (C) and a “variable” (V) region. The V regions determine the antigen binding specificity of the antibody, whilst the C regions provide structural support and function in non-antigen-specific interactions with immune effectors. The antigen binding specificity of an antibody or antigen-binding fragment of an antibody is the ability of an antibody to specifically bind to a particular antigen.

The antigen binding specificity of an antibody is determined by the structural characteristics of the V region. The variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Each V region typically comprises three complementarity determining regions (“CDRs”, each of which contains a “hypervariable loop”), and four framework regions. An antibody binding site, the minimal structural unit required to bind with substantial affinity to a particular desired antigen, will therefore typically include the three CDRs, and at least three, preferably four, framework regions interspersed therebetween to hold and present the CDRs in the appropriate conformation. Classical four chain antibodies have antigen binding sites which are defined by VH and VL domains in cooperation. Certain antibodies, such as camel and shark antibodies, lack light chains and rely on binding sites formed by heavy chains only. Single domain engineered immunoglobulins can be prepared in which the binding sites are formed by heavy chains or light chains alone, in absence of cooperation between VH and VL.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β3-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody that are responsible for antigen binding. The hypervariable region may comprise amino acid residues from a “complementarity determining region” or “CDR” (e.g., around about residues 24-34 (L1), 50-56 (L2) and 89-97 (L3) in the VL, and around about 31-35B (H1), 50-65 (H2) and 95-102 (H3) in the VH (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the VL, and 26-32 (H1), 52A-55 (H2) and 96-101 (H3) in the VH (Chothia and Lesk J. Mol. Biol. 196:901-917 (1987)).

“Framework” or “FR” residues are those variable domain residues other than the hypervariable region residues as herein defined.

“Hinge region” in the context of an antibody or half-antibody is generally defined as stretching from Glu216 to Pro230 of human IgG1 (Burton, Molec. Immunol.22:161-206 (1985)). Hinge regions of other IgG isotypes may be aligned with the IgG1 sequence by placing the first and last cysteine residues forming inter-heavy chain S—S bonds in the same positions.

The “lower hinge region” of an Fc region is normally defined as the stretch of residues immediately C-terminal to the hinge region, i.e. residues 233 to 239 of the Fc region. Prior to the present application, FcyR binding was generally attributed to amino acid residues in the lower hinge region of an IgG Fc region.

The “CH2 domain” of a human IgG Fc region usually extends from about residues 231 to about 340 of the IgG. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22:161-206 (1985).

The “CH3 domain” comprises the stretch of residues C-terminal to a CH2 domain in an Fc region (i.e. from about amino acid residue 341 to about amino acid residue 447 of an IgG).

“Antibody fragments” comprise a portion of an intact antibody, preferably comprising the antigen binding region thereof. Examples of antibody fragments include Fab, Fab′, F(ab′)2, and Fv fragments; diabodies; tandem diabodies (taDb), linear antibodies (e.g., U.S. Pat. No. 5,641,870, Example 2; Zapata et al., Protein Eng. 8(10):1057-1062 (1995)); one-armed antibodies, single variable domain antibodies, minibodies, single-chain antibody molecules; multispecific antibodies formed from antibody fragments (e.g., including but not limited to, db-Fc, taDb-Fc, taDb-CH3, (scFV)4-Fc, di-scFv, bi-scFv, or tandem (di,tri)-scFv); and Bi-specific T-cell engagers (BiTEs).

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Fv” is the minimum antibody fragment that contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa (K) and lambda (2), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgGl, IgG2, IgG3, IgG4, IgA, and IgA2. The heavy chain constant domains that correspond to the different classes of antibodies are called a, 6, c, y, and μ, respectively. The subunit structures and three-dimensional configurations of different classes of immunoglobulins are well known.

“Single-chain Fv” or “scFv” antibody fragments comprise the VH and VL domains of antibody, wherein these domains are present in a single polypeptide chain. In some embodiments, the Fv polypeptide further comprises a polypeptide linker between the VH and VL domains that enables the scFv to form the desired structure for antigen binding. For a review of scFv see Pliickthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “half-antibody” or “hemimer” as used herein refers to a monovalent antigen binding polypeptide. In certain embodiments, a half antibody or hemimer comprises a VH/VL unit and optionally at least a portion of an immunoglobulin constant domain. In certain embodiments, a half antibody or hemimer comprises one immunoglobulin heavy chain associated with one immunoglobulin light chain, or an antigen binding fragment thereof. In certain embodiments, a half antibody or hemimer is mono-specific, i.e., binds to a single antigen or epitope. One skilled in the art will readily appreciate that a half-antibody may have an antigen binding domain consisting of a single variable domain, e.g., originating from a camelidae.

The term “VH/VL unit” refers to the antigen-binding region of an antibody that comprises at least one VH HVR and at least one VL HVR. In certain embodiments, the VH/VL unit comprises at least one, at least two, or all three VH HVRs and at least one, at least two, or all three VL HVRs. In certain embodiments, the VH/VL unit further comprises at least a portion of a framework region (FR). In some embodiments, a VH/VL unit comprises three VH HVRs and three VL HVRs. In some such embodiments, a VH/VL unit comprises at least one, at least two, at least three or all four VH FRs and at least one, at least two, at least three or all four VL FRs.

The term “multispecific antibody” is used in the broadest sense and specifically covers an antibody comprising an antigen-binding domain that has polyepitopic specificity (i.e., is capable of specifically binding to two, or more, different epitopes on one biological molecule or is capable of specifically binding to epitopes on two, or more, different biological molecules). In some embodiments, an antigen-binding domain of a multispecific antibody (such as a bispecific antibody or a divalent F(ab′)2) comprises two VH/VL units, wherein a first VH/VL unit specifically binds to a first epitope and a second VH/VL unit specifically binds to a second epitope, wherein each VH/VL unit comprises a heavy chain variable domain (VH) and a light chain variable domain (VL). Such multispecific antibodies include, but are not limited to, full length antibodies, antibodies having two or more VL and VH domains, antibody fragments such as Fab, Fv, dsFv, scFv, diabodies, bispecific diabodies and triabodies, antibody fragments that have been linked covalently or non-covalently. A VH/VL unit that further comprises at least a portion of a heavy chain constant region and/or at least a portion of a light chain constant region may also be referred to as a “hemimer” or “half antibody.” In some embodiments, a half antibody comprises at least a portion of a single heavy chain variable region and at least a portion of a single light chain variable region. In some such embodiments, a bispecific antibody that comprises two half antibodies and binds to two antigens comprises a first half antibody that binds to the first antigen or first epitope but not to the second antigen or second epitope and a second half antibody that binds to the second antigen or second epitope and not to the first antigen or first epitope. According to some embodiments, the multispecific antibody is an IgG antibody that binds to each antigen or epitope with an affinity of 5 M to 0.001 pM, 3 M to 0.001 pM, 1 M to 0.001 pM, 0.5 M to 0.001 pM, or 0.1 M to 0.001 pM. In some embodiments, a hemimer comprises a sufficient portion of a heavy chain variable region to allow intramolecular disulfide bonds to be formed with a second hemimer. In some embodiments, a hemimer comprises a knob mutation or a hole mutation, for example, to allow heterodimerization with a second hemimer or half antibody that comprises a complementary hole mutation or knob mutation. Knob mutations and hole mutations are discussed further below.

A “bispecific antibody” is a multispecific antibody comprising an antigen-binding domain that is capable of specifically binding to two different epitopes on one biological molecule or is capable of specifically binding to epitopes on two different biological molecules. A bispecific antibody may also be referred to herein as having “dual specificity” or as being “dual specific.” Unless otherwise indicated, the order in which the antigens bound by a bispecific antibody are listed in a bispecific antibody name is arbitrary. In some embodiments, a bispecific antibody comprises two half antibodies, wherein each half antibody comprises a single heavy chain variable region and optionally at least a portion of a heavy chain constant region, and a single light chain variable region and optionally at least a portion of a light chain constant region. In certain embodiments, a bispecific antibody comprises two half antibodies, wherein each half antibody comprises a single heavy chain variable region and a single light chain variable region and does not comprise more than one single heavy chain variable region and does not comprise more than one single light chain variable region. In some embodiments, a bispecific antibody comprises two half antibodies, wherein each half antibody comprises a single heavy chain variable region and a single light chain variable region, and wherein the first half antibody binds to a first antigen and not to a second antigen and the second half antibody binds to the second antigen and not to the first antigen.

The term “knob-into-hole” or “KiH” technology as used herein refers to the technology directing the pairing of two polypeptides together in vitro or in vivo by introducing a protuberance (knob) into one polypeptide and a cavity (hole) into the other polypeptide at an interface in which they interact. For example, KiHs have been introduced in the Fc:Fc binding interfaces, CL:CH1 interfaces or VH/VL interfaces of antibodies (see, e.g., US 2011/0287009, US2007/0178552, WO 96/027011, WO 98/050431, and Zhu et al., 1997, Protein Science 6:781-788). In some embodiments, KiHs drive the pairing of two different heavy chains together during the manufacture of multispecific antibodies. For example, multispecific antibodies having KiH in their Fc regions can further comprise single variable domains linked to each Fc region, or further comprise different heavy chain variable domains that pair with similar or different light chain variable domains. KiH technology can also be used to pair two different receptor extracellular domains together or any other polypeptide sequences that comprises different target recognition sequences (e.g., including affibodies, peptibodies and other Fc fusions).

The term “knob mutation” as used herein refers to a mutation that introduces a protuberance (knob) into a polypeptide at an interface in which the polypeptide interacts with another polypeptide. In some embodiments, the other polypeptide has a hole mutation (see e.g., U.S. Pat. Nos. 5,731,168, 5,807,706, 5,821,333, 7,695,936, 8,216,805, each incorporated herein by reference in its entirety).

The term “hole mutation” as used herein refers to a mutation that introduces a cavity (hole) into a polypeptide at an interface in which the polypeptide interacts with another polypeptide. In some embodiments, the other polypeptide has a knob mutation (see e.g., U.S. Pat. Nos. 5,731,168, 5,807,706, 5,821,333, 7,695,936, 8,216,805, each incorporated herein by reference in its entirety).

The expression “single domain antibodies” (sdAbs) or “single variable domain (SVD) antibodies” generally refers to antibodies in which a single variable domain (VH or VL) can confer antigen binding. In other words, the single variable domain does not need to interact with another variable domain in order to recognize the target antigen. Examples of single domain antibodies include those derived from camelids (lamas and camels) and cartilaginous fish (e.g., nurse sharks) and those derived from recombinant methods from humans and mouse antibodies (Nature (1989) 341:544-546; Dev Comp Immunol (2006) 30:43-56; Trend Biochem Sci (2001) 26:230-235; Trends Biotechnol (2003):21:484-490; WO 2005/035572; WO 03/035694; FEBS Lett (1994) 339:285-290; WO00/29004; WO 02/051870).

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the methods provided herein may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

For the purposes herein, an “intact antibody” is one comprising heavy and light variable domains as well as an Fc region. The constant domains may be native sequence constant domains (e.g. human native sequence constant domains) or amino acid sequence variant thereof. Preferably, the intact antibody has one or more effector functions.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

A “naked antibody” is an antibody (as herein defined) that is not conjugated to a heterologous molecule, such as a cytotoxic moiety or radiolabel.

As used herein, the term “immunoadhesin” designates molecules which combine the binding specificity of a heterologous protein (an “adhesin”) with the effector functions of immunoglobulin constant domains. Structurally, the immunoadhesins comprise a fusion of an amino acid sequence with a desired binding specificity, which amino acid sequence is other than the antigen recognition and binding site of an antibody (i.e., is “heterologous” compared to a constant region of an antibody), and an immunoglobulin constant domain sequence (e.g., CH2 and/or CH3 sequence of an IgG). Exemplary adhesin sequences include contiguous amino acid sequences that comprise a portion of a receptor or a ligand that binds to a protein of interest. Adhesin sequences can also be sequences that bind a protein of interest, but are not receptor or ligand sequences (e.g., adhesin sequences in peptibodies). Such polypeptide sequences can be selected or identified by various methods, include phage display techniques and high throughput sorting methods. The immunoglobulin constant domain sequence in the immunoadhesin can be obtained from any immunoglobulin, such as IgG-1, IgG-2, IgG-3, or IgG-4 subtypes, IgA (including IgA-1 and IgA-2), IgE, IgD, or IgM.

The terms “Fc receptor” or “FcR” are used to describe a receptor that binds to the Fc region of an antibody. In some embodiments, the FcR is a native sequence human FcR. Moreover, a preferred FcR is one that binds an IgG antibody (a gamma receptor) and includes receptors of the FcyRI, FcyRII, and Fcy RIII subclasses, including allelic variants and alternatively spliced forms of these receptors. FcyRII receptors include FcyRIIA (an “activating receptor”) and FcyRIIB (an “inhibiting receptor”), which have similar amino acid sequences that differ primarily in the cytoplasmic domains thereof. Activating receptor FcyRIIA contains an immunoreceptor tyrosine-based activation motif (ITAM) in its cytoplasmic domain. Inhibiting receptor FcyRIIB contains an immunoreceptor tyrosine-based inhibition motif (ITIM) in its cytoplasmic domain. (see Daeron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Chn. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

“Impurities” refer to materials that are different from the desired polypeptide product. The impurity may refer to product-specific polypeptides such as one-armed antibodies and misassembled antibodies, antibody variants including basic variants and acidic variants, and aggregates. Other impurities include process specific impurities including without limitation: host cell materials such as host cell protein (HCP); leached Protein A; nucleic acid; another polypeptide; endotoxin; viral contaminant; cell culture media component, etc. In some examples, the impurity may be an HCP from, for example but not limited to, a bacterial cell such as an E. coli cell (ECP), an insect cell, a prokaryotic cell, a eukaryotic cell, a yeast cell, a mammalian cell, an avian cell, a fungal cell. In some examples, the impurity may be an HCP from a mammalian cell, such as a CHO cell, i.e., a CHO cell protein (CHOP). The impurity may refer to accessory proteins used to facilitate expression, folding or assembly of multispecific antibodies; for example, prokaryotic chaperones such as FkpA, DsbA and DsbC.

“Complex” or “complexed” as used herein refers to the association of two or more molecules that interact with each other through bonds and/or forces (e.g., van der waals, hydrophobic, hydrophilic forces) that are not peptide bonds. In one embodiment, the complex is heteromultimeric. It should be understood that the term “protein complex” or “polypeptide complex” as used herein includes complexes that have a non-protein entity conjugated to a protein in the protein complex (e.g., including, but not limited to, chemical molecules such as a toxin or a detection agent).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. In certain embodiments, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al., Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “sequential” as used herein with regard to chromatography refers to chromatography steps in a specific sequence; e.g., a first chromatography step followed by a second chromatography step followed by a third chromatography step, etc. Additional steps may be included between the sequential chromatography steps.

The term “continuous” as used herein with regard to chromatography refers to having a first chromatography material and a second chromatography material either directly connected or some other mechanism which allows for continuous flow between the two chromatography materials.

“Loading density” refers to the amount, e.g. grams, of composition put in contact with a volume of chromatography material, e.g. liters. In some examples, loading density is expressed in g/L.

A “sample” refers to a small portion of a larger quantity of material. Generally, testing according to the methods described herein is performed on a sample. The sample is typically obtained from a recombinant polypeptide preparation obtained, for example, from cultured recombinant polypeptide-expressing cell lines, also referred to herein as “product cell lines,” or from cultured host cells. As used herein, “host cells” do not contain genes for the expression of recombinant polypeptides of interest or products. A sample may be obtained from, for example but not limited to, harvested cell culture fluid, from an in-process pool at a certain step in a purification process, or from the final purified product. The sample may also include diluents, buffers, detergents, and contaminating species, debris and the like that are found mixed with the desired molecule (such as a multispecific antibody, e.g., a bispecific antibody).

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

Methods of Purification of a Multispecific Antibody

The reliability of the antibody manufacturing process has been improved by a number of strategies including, but not limited to expressing “knob-in-hole” multispecific antibodies as well as the development of CrossMab antibodies. The incorporation of such strategies for manufacture of multispecific antibodies in single cells, however, continues to rely on downstream purification processes to eliminate antibody variants comprising misparied polypeptides.

In certain non-limiting embodiments, the present disclosure provides methods for purifying a multispecific antibody. In certain embodiments, the multispecific antibody is a CrossMab antibody. In certain embodiments, the multispecific antibody is a bispecific antibody. In certain embodiments, the multispecific antibody is a divalent F(ab′)2 that comprises a first F(ab) that binds a first target and a second F(ab) that binds a second target. In certain embodiments, the multispecific antibody is a dual specific antibody, i.e. an antibody having two antigen-binding arms that are identical in amino acid sequence, and wherein each Fab arm is capable of recognizing two antigens (such as a dual action Fab antibody).

In certain embodiments, the purification of the multispecific antibody comprises a multi-mode chromatography. In some embodiments, the multispecific antibody is assembled before capture chromatography. In some embodiments, the multispecific antibody is assembled after capture chromatography.

In certain embodiments, the multispecific antibody (such as a bispecific antibody or a divalent F(ab′)₂) comprises two or more antibody arms wherein different antibody arms bind different epitopes. In certain embodiments, the different epitopes are on the same antigen. In certain embodiments, the different epitopes are on different antigens. In certain embodiments, antibody arms comprise VH/VL units. In certain embodiments, the antibody arms comprise hemimers, also known as half-antibodies. In certain embodiments, the heavy chain of one antibody arm is modified to comprise a “knob” and the heavy chain of another antibody arm comprises a “hole” such that the knob of the first heavy chain fits into the hole of the second heavy chain.

In certain embodiments, the multispecific antibody is produced in the same host cell. For example, the following listing includes the product-related variants identified in a CrossMab bispecific antibody culture harvest where multispecific antibody is produced in the same host cell and the harvest is purified by protein A affinity chromatography.

Multispecific Antibody Variant

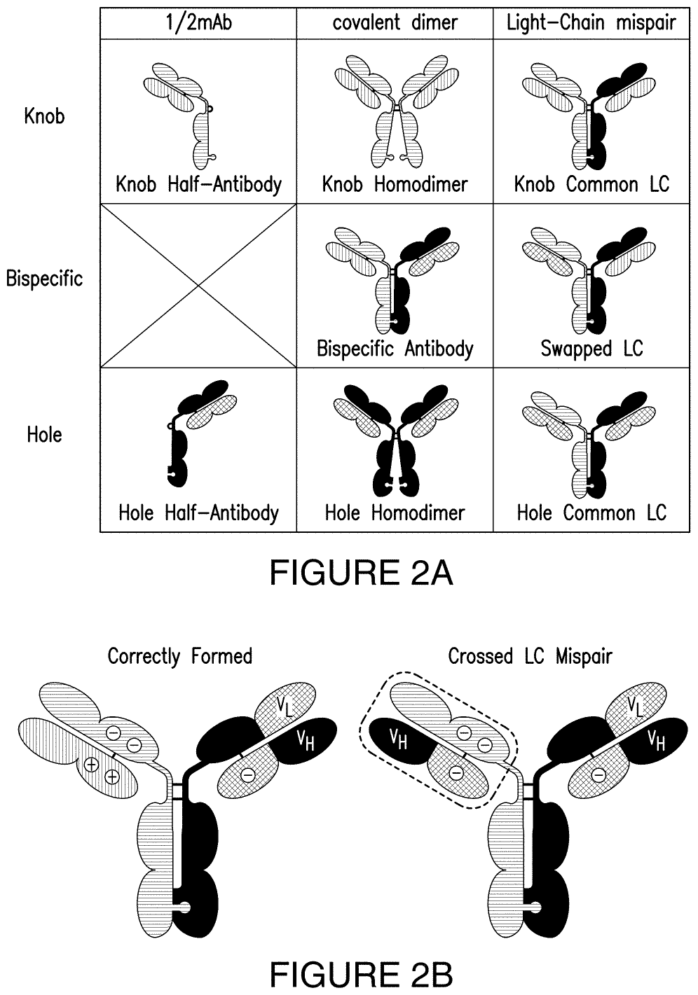

aAgA knob half-antibody
aAgBhole half antibody
aAgA-aAgA knob-knob homodimer
LC-mispaired Bispecific (aAgA common LC)
Bispecific antibody (correctly formed, see FIGS. 2A and 2B)
LC-mispaired Bispecific (aAgB crossed LC, see FIGS. 2A and 2B)
aAgB-aAgB hole-hole homodimer

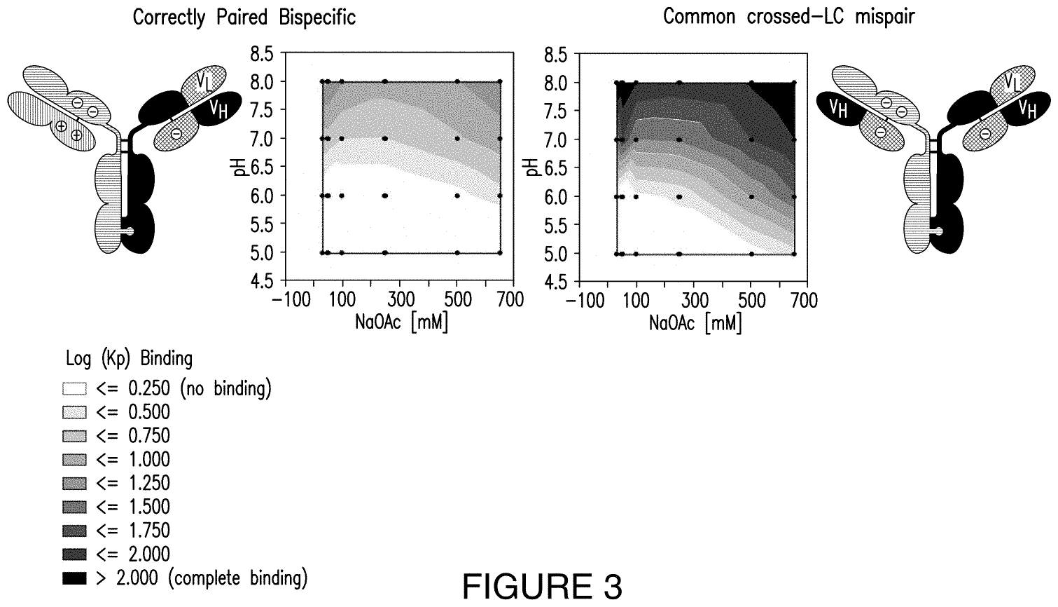

An automated liquid-handling system was used to test binding of the feedstock to five different chromatography resins under a variety of pH and buffer-strength conditions. Following incubation, the unbound fraction was analyzed and it was surprisingly observed that depletion of LC mispaired variant (described in FIGS. 2A and 2B) only occurred under conditions simultaneously promoting anion-exchange behavior (high pH) and hydrophobic binding (high salt concentration) depicted in FIG. 3.

In certain embodiments, the cell culture medium is collected and the antibodies are subjected to a multi-mode chromatography. In certain embodiments, the multi-mode material comprises functional groups capable of one of more of the following functionalities: anionic exchange, cationic exchange, hydrogen bonding, pi-pi bond interactions, hydrophilic interactions, thiophilic interactions, and hydrophobic interactions. In certain embodiments, the multi-mode material comprises functional groups capable of anionic exchange and hydrophobic interactions.

In certain embodiments, the multi-mode material comprises a positively charged group and an aromatic ring structure. In certain embodiments, the positively charged group is an amine or a quaternary ammonium ion. In certain embodiments, the aromatic ring structure is a benzyl-group. In certain embodiments, the multi-mode material comprises N-benzyl-N-methyl ethanolamine, N, N-dimethyl benzylamine, 4-mercapto-ethyl-pyridine, 2-benzamido-4-mercaptobutanoic acid, hexylamine, phenylpropylamine, cross-linked polyallylamine, or a combination thereof. For example, but without any limitation, the multi-mode materials include Capto™ Adhere resin, Capto™ MMC resin, MEP HyperCel™ resin, HEA HyperCel™ resin, PPA HyperCel™ resin, Eshmuno® HCX, Capto™ Adhere ImpRes, Capto™ MMC Impres, Nuvia™ cPrime™ membrane. In certain embodiments, the multi-mode material is Capto™ Adhere resin. In certain embodiments, the multi-mode material is Capto™ MMC. In certain embodiments, the multi-mode material is in a column. In certain embodiments, the multi-mode material is in a membrane. In certain embodiments, the multi-mode chromatography is performed in “bind and elute” mode. In certain embodiments, the multi-mode chromatography is performed in “flow through” mode.

In certain embodiments, the elution is a step elution. In certain embodiments, the elution is gradient elution.

In certain embodiments, the methods disclosed herein further comprise a capture chromatography. In certain embodiments, the capture chromatography is affinity chromatography. In certain embodiments, the affinity chromatography is Protein A chromatography. In certain embodiments, the affinity chromatography is Protein G chromatography. In certain embodiments, the affinity chromatography is Protein A/G chromatography. In certain embodiments, the affinity chromatography is Protein L chromatography. Following capture chromatography, purified antibody arms may be analyzed; for example, by SDS-PAGE, SEC chromatography, mass spectrometry, etc.

In certain embodiments, the cell culture medium is collected and subjected to a capture chromatography. In certain embodiments, the eluate from the affinity chromatography step is subsequently applied to a multi-mode chromatography disclosed herein. In certain embodiments, the affinity chromatography includes, for example but without any limitation, protein A chromatography, protein G chromatography, protein A/G chromatography, or protein L chromatography. In certain embodiments, the affinity chromatography material includes, for example and without any limitation, ProSep®-vA, ProSep® Ultra Plus, Protein A Sepharose™ Fast Flow, Toyopearl™ AF-rProtein A, MabSelect™, Mab Select SuRe™, Mab Select SuRe™ LX, KappaSelect, CaptureSelect™, and CaptureSelect™ FcXL. In certain embodiments, the affinity chromatography material is in a column. In certain embodiments, the affinity chromatography is performed in “bind and elute mode” (alternatively referred to as “bind and elute process”). “Bind and elute mode” refers to a product separation technique in which a product (such as the multispecific antibody) in the sample binds the affinity chromatography material and is subsequently eluted from the affinity chromatography material. In certain embodiments, the elution is a step elution, in which the composition of the mobile phase is changed stepwise, at one or several occasions, during the elution process. In certain embodiments, the elution is gradient elution, in which the composition of the mobile phase is changed continuously during the elution process. In certain embodiments, the affinity chromatography material is a membrane. In certain embodiments, the affinity chromatography is protein A chromatography. In certain embodiments, the protein A chromatography is MAbSelect™ SuRe chromatography. In certain embodiments, the affinity chromatography is CaptureSelect™ chromatography. In certain embodiments, the affinity chromatography is CaptureSelect™ FcXL chromatography.

In certain embodiments, the capture chromatography and the multi-mode chromatography are continuous, e.g., wherein the capture chromatography material and the multi-mode material are either directly connected or connected by some other mechanism that allows for continuous flow between the capture chromatography material and the multi-mode material. In certain embodiments, the capture chromatography and the multi-mode chromatography are contiguous, wherein the multi-mode chromatography is performed directly after the capture chromatography.

In certain embodiments, the eluate from the capture chromatography is subject to one or more additional chromatography steps prior being applied to the multi-mode resin. In certain non-limiting embodiments, for example, the eluate from the capture chromatography can be subject to any one or more of the following chromatography steps in any order and/or in any combination prior to being subject to a multi-mode chromatography: hydrophobic interaction (HIC) chromatography, anion exchange chromatography, cation exchange chromatography, size exclusion chromatography, affinity chromatography, ceramic hydroxyapatite (CHT) chromatography, hydrophilic interaction liquid chromatography (HILIC), etc.

Hydrophobic interaction chromatography is a liquid chromatography technique that separates biomolecules according to hydrophobicity. For example, but without any limitation, HIC chromatography materials include Toyopearl™ Hexyl-650, Toyopearl™ Butyl-650, Toyopearl™ Phenyl-650, Toyopearl™ Ether-650, HiTrap® Sepharose, Octyl Sepharose®, Phenyl Sepharose™ or Butyl Sepharose™. In certain embodiments, the HIC chromatography material comprises phenyl sepharose. In certain embodiments, the HIC chromatography is performed in “bind and elute” mode. In certain embodiments, the HIC chromatography is performed in “flow through” mode. In certain embodiments, the HIC chromatography material is in a column. In certain embodiments, the HIC chromatography material is in a membrane.

Anion exchange chromatography material is a solid phase that is positively charged and has free anions for exchange with anions in an aqueous solution (such as a composition comprising a multispecific antibody and an impurity) that is passed over or through the solid phase. In certain embodiments, the anion exchange material can be a membrane, a monolith, or resin. In certain embodiments, the anion exchange material can be a resin. In certain embodiments, the anion exchange material can comprise a primary amine, a secondary amine, a tertiary amine or a quaternary ammonium ion functional group, a polyamine functional group, or a diethylaminoaethyl functional group. For example, but without any limitation, anion exchange materials include Poros™ HQ 50, Poros™ PI 50, Poros™ D, Mustang™ Q, Q Sepharose™ Fast Flow (QSFF), Accell™ Plus Quaternary Methyl Amine (QMA) resin, Sartobind STIC®, and DEAE-Sepharose™. In certain embodiments, the anion exchange chromatography is performed in “bind and elute” mode. In certain embodiments, the anion exchange chromatography is performed in “flow through” mode. In certain embodiments, the anion exchange chromatography material is in a column. In certain embodiments, the anion exchange chromatography material is a membrane.

Cation exchange chromatography material is a solid phase that is negatively charged and has free cations for exchange with cations in an aqueous solution (such as a composition comprising a multispecific antibody and an impurity) that is passed over or through the solid phase. In certain embodiments, the cation exchange material may be a membrane, a monolith, or resin. In certain embodiments, the cation exchange material may be a resin. The cation exchange material can comprise a carboxylic acid functional group or a sulfonic acid functional group. For example, but without any limitation, the cation exchange material can include sulfonate, carboxylic, carboxymethyl sulfonic acid, sulfoisobutyl, sulfoethyl, carboxyl, sulphopropyl, sulphonyl, sulphoxyethyl, or orthophosphate. In certain embodiments, the cation exchange chromatography material is a cation exchange chromatography column. In certain embodiments of the above, the cation exchange chromatography material is a cation exchange chromatography membrane. For example, but without any limitation, cation exchange materials include Mustang™ S, Sartobind® S, SO3 Monolith (such as, e.g., CIM®, ClMmultus® and CIMac® SO3), S Ceramic HyperD®, Poros™ XS, Poros™ HS 50, Poros™ HS 20, sulphopropyl-Sepharose® Fast Flow (SPSFF), SP-Sepharose® XL (SPXL), CM Sepharose™ Fast Flow, Capto™ S, Fractogel™ EMD Se Hicap, Fractogel™ EMD S03, or Fractogel™ EMD COO. In certain embodiments, the cation exchange chromatography is performed in “bind and elute” mode. In certain embodiments, the cation exchange chromatography is performed in “flow through” mode. In certain embodiments of the above, the cation exchange chromatography material is in a column. In certain embodiments of the above, the cation exchange chromatography material is in a membrane.

In certain embodiments, the present disclosure provides methods of separating a multispecific antibody, i.e., a bispecific antibody, from a composition comprising said multispecific antibody and an impurity, the method comprising subjecting the composition to a multi-mode chromatography, and collecting a fraction comprising the multispecific antibody, wherein the multispecific antibody is produced by the same host cell. In certain embodiments, the multi-mode chromatography is performed in “bind and elute” mode.

In certain embodiments, the method comprises subjecting the composition to a capture chromatography to produce a first eluate, subjecting the first eluate to a multi-mode chromatography, and collecting a fraction comprising the multispecific antibody. In certain embodiments, the capture chromatography is a protein A chromatography.

In certain embodiments, the eluate from the multi-mode chromatography is subject to one or more additional chromatography steps. For example, but without any limitation, the eluate from the multi-mode chromatography can be subject to any one or more of the following chromatography steps in any order and/or in any combination: hydrophobic interaction (HIC) chromatography, anion exchange chromatography, cation exchange chromatography, size exclusion chromatography, affinity chromatography, ceramic hydroxyapatite (CHT) chromatography, hydrophilic interaction liquid chromatography (HILIC), multi-mode chromatography, etc.

In certain embodiments, the methods comprise using a buffer. Various buffers can be employed during the purification of the multispecific antibody. For example, buffers can have a different pH and/or conductivity based on the characteristics of the multispecific antibody. In certain embodiments, the buffer can be a loading buffer, an equilibration buffer, or a wash buffer. In certain embodiments, one or more of the loading buffer, the equilibration buffer, and/or the wash buffer are the same. In certain embodiments, the loading buffer, the equilibration buffer, and/or the wash buffer are different. In certain embodiments, the buffer comprises a salt. In certain embodiments, the buffer comprises sodium chloride, sodium acetate, Tris HCl, Tris acetate, sodium phosphate, potassium phosphate, IVIES, CHES, MOPS, BisTris, arginine, arginine HCl, or a mixture thereof. In certain embodiments, the buffer is a sodium chloride buffer. In some embodiments, the buffer is a sodium acetate buffer. In certain embodiments, the buffer is Tris, arginine, phosphate, IVIES, CHES, or MOPS buffer.

“Load” refers to the composition being loaded onto a chromatography material. Loading buffer is the buffer used to load the composition (e.g., a composition comprising a multispecific antibody and an impurity) onto a chromatography material (such as any one of the chromatography materials described herein). The chromatography material can be equilibrated with an equilibration buffer prior to loading the composition to be purified. The wash buffer is used after loading the composition onto a chromatography material. An elution buffer is used to elute the polypeptide of interest from the solid phase.

Loading of a composition comprising the multispecific antibody (such as a composition comprising the multispecific antibody and an impurity) on any of the chromatography materials described herein may be optimized for separation of the multispecific antibody from the impurity. In certain embodiments, loading of the composition comprising the multispecific antibody (such as a composition comprising the multispecific antibody and an impurity) onto the chromatography material is optimized for binding of the multispecific antibody to the chromatography material when the chromatography is performed in bind and elute mode (e.g., multi-mode chromatography).

Conductivity refers to the ability of an aqueous solution to conduct an electric current between two electrodes. In solution, the current flows by ion transport. Therefore, with an increasing amount of ions present in the aqueous solution, the solution will have a higher conductivity. The basic unit of measure for conductivity is the Siemen (mS/cm) or ohms (mho), and can be measured using a conductivity meter, such as various models of Orion conductivity meters. Since electrolytic conductivity is the capacity of ions in a solution to carry electrical current, the conductivity of a solution can be altered by changing the concentration of ions therein. In certain non-limiting embodiments, for example, the concentration of a buffering agent and/or the concentration of a salt (e.g. sodium chloride, sodium acetate, or potassium chloride) in the solution can be altered in order to achieve the desired conductivity. In certain embodiments, the salt concentration of the various buffers is modified to achieve the desired conductivity.

In certain non-limiting embodiments, for example, the composition comprising the multispecific antibody (such as a composition comprising the multispecific antibody and an impurity) is loaded onto the chromatography material in a loading buffer at a number of different pH values while the conductivity of the loading buffer is constant. In certain embodiments, the solution comprising the multispecific antibody is loaded onto the chromatography material in a loading buffer at a number of different conductivities while the pH of the loading buffer is constant. Upon completion of loading the composition comprising the multispecific antibody (such as a composition comprising the multispecific antibody and an impurity) on the chromatography material and elution of the multispecific antibody from the chromatography material into a pool fraction, the amount of impurity remaining in the pool fraction provides information regarding the separation of the multispecific antibody from the impurity for a given pH or conductivity. Similarly, for chromatography where the multispecific antibody flows through the chromatography material the loading buffer is optimized for pH and conductivity such that the multispecific antibody flows through the chromatography whereas the impurity is retained by the chromatography material or flows through the chromatography material at a different rate than the multispecific antibody.

In certain embodiments, the loading density of the solution comprising the multispecific antibody is greater than about any of 1 g/L, 5 g/L, 10 g/L, 20 g/L, 30 g/L, 40 g/L, 50 g/L, 60 g/L, 70 g/L, 80 g/L, 90 g/L, 100 g/L, 110 g/L, 120 g/L, 130 g/L, 140 g/L, or 150 g/L of the affinity chromatography material. In certain embodiments, the loading density of the solution comprising the multispecific antibody is between about any of 1 g/L and 5 g/L, 5 g/L and 10 g/L, 10 g/L and 20 g/L, 20 g/L and 30 g/L, 30 g/L and 40 g/L, 40 g/L and 50 g/L, 50 g/L and 60 g/L, 60 g/L and 70 g/L, 70 g/L and 80 g/L, 80 g/L and 90 g/L, 90 g/L and 100 g/L, of the capture chromatography material.

In certain embodiments, the eluate obtained following the capture chromatography is loaded onto a multi-mode chromatography material (e.g., Capto™ Adhere). In certain embodiments, the eluate obtained following the capture chromatography is loaded onto a multi-mode chromatography material at a loading density of the multispecific antibody of greater than about any of 10 g/L, 20 g/L, 30 g/L, 40 g/L, 50 g/L, 60 g/L, 70 g/L, 80 g/L, 90 g/L, 100 g/L, 110 g/L, 120 g/L, 130 g/L, 140 g/L, or 150 g/L of the multi-mode chromatography material. In certain embodiments, the eluate obtained following the capture chromatography is loaded onto a multi-mode chromatography material at a loading density of the multispecific antibody between about any of 1 g/L and 5 g/L, 5 g/L and 10 g/L, 10 g/L and 20 g/L, 20 g/L and 30 g/L, 30 g/L and 40 g/L, 40 g/L and 50 g/L, 50 g/L and 60 g/L, 60 g/L and 70 g/L, 70 g/L and 80 g/L, 80 g/L and 90 g/L, 90 g/L and 100 g/L of the multi-mode chromatography material.

In certain embodiments, the eluate obtained following the multi-mode chromatography is loaded onto a subsequent chromatography material (such as a hydrophobic interaction (HIC) chromatography material, anion exchange chromatography material, cation exchange chromatography material, size exclusion chromatography material, affinity chromatography material, or an additional multi-mode chromatography material) at a loading density of the multispecific antibody of greater than about any of 30 g/L, 40 g/L, 50 g/L, 60 g/L, 70 g/L, 80 g/L, 90 g/L, 100 g/L, 110 g/L, 120 g/L, 130 g/L, 140 g/L, or 150 g/L of the subsequent chromatography material. In some embodiments, the eluate obtained following the multi-mode chromatography is loaded onto the subsequent chromatography material (such as a hydrophobic interaction (HIC) chromatography material, anion exchange chromatography material, cation exchange chromatography material, size exclusion chromatography material, affinity chromatography material, or an additional multi-mode chromatography material) at a loading density of the multispecific antibody between about any of 10 g/L and 20 g/L, 20 g/L and 30 g/L, 30 g/L and 40 g/L, 40 g/L and 50 g/L, 50 g/L and 60 g/L, 60 g/L and 70 g/L, 70 g/L and 80 g/L, 80 g/L and 90 g/L, 90 g/L and 100 g/L, of the subsequent chromatography material.

Elution, as used herein, is the removal of the product, e.g. multispecific antibody, from the chromatography material. Elution buffer is the buffer used to elute the multispecific antibody from a chromatography material. In certain embodiments, the elution buffer has a lower conductivity than the loading buffer. In certain embodiments, the elution buffer has a higher conductivity than the loading buffer. In certain embodiments, the elution buffer has a lower pH than the load buffer. In certain embodiments, the elution buffer has a higher pH than the load buffer. In certain embodiments, the elution buffer has a different conductivity and a different pH than the load buffer.

In certain embodiments, elution of the multispecific antibody from the chromatography material is optimized for yield of product with minimal impurity and at minimal elution volume or pool volume. In certain non-limiting embodiments, for example, the composition comprising the multispecific antibody can be loaded onto the chromatography material in a loading buffer. Upon completion of load, the multispecific antibody is eluted with buffers at a number of different pH values while the conductivity of the elution buffer is constant. Alternatively, the multispecific antibody can be eluted from the chromatography material in an elution buffer at a number of different conductivities while the pH of the elution buffer is constant. Upon completion of elution of the multispecific antibody from the chromatography material, the amount of an impurity in the pool fraction provides information regarding the separation of the multispecific antibody or antibody arm from the impurities for a given pH or conductivity. Elution of the multispecific antibody in a high number of column volumes (e.g. eight column volumes) indicates “tailing” of the elution profile.

In certain embodiments, the method disclosed herein comprise use of buffers. Various buffers which can be employed based on the desired pH of the buffer, the desired conductivity of the buffer, the characteristics of the protein of interest, the chromatography material, and the purification process (e.g., “bind and elute” or “flow through” mode). In certain embodiments, the methods comprise the use of at least one buffer. In certain embodiments, the buffer can be a loading buffer, an equilibration buffer, an elution buffer, or a wash buffer. In certain embodiments, one or more of the loading buffer, the equilibration buffer, the elution buffer and/or the wash buffer are the same. In certain embodiments, the loading buffer, the equilibration buffer, and/or the wash buffer are different. In certain embodiments, the buffer comprises a salt. In certain embodiments, the loading buffer can comprise sodium chloride, sodium acetate, Tris, arginine, phosphate, MOPS, IVIES, CHES, BisTris, ammonium sulfate, sodium sulfate, citrate, succinate, or mixtures thereof. In certain embodiments, the buffer is a sodium chloride buffer. In certain embodiments, the buffer is a sodium acetate buffer. In certain embodiments, the buffer is Tris, arginine, phosphate, MES, CHES, or MOPS buffer. In certain embodiments, the buffer comprises Tris. In certain embodiments, the buffer comprises arginine.

In certain embodiments, the loading buffer has a conductivity of greater than about any of 1.0 mS/cm, 1.5 mS/cm, 2.0 mS/cm, 2.5 mS/cm, 3.0 mS/cm, 3.5 mS/cm, 4.0 mS/cm, 4.5 mS/cm, 5.0 mS/cm, 5.5 mS/cm, 6.0 mS/cm, 6.5 mS/cm, 7.0 mS/cm, 7.5 mS/cm, 8.0 mS/cm, 8.5 mS/cm, 9.0 mS/cm, 9.5 mS/cm, 10 mS/cm or 20 mS/cm. In certain embodiments, the conductivity can be between about any of 1 mS/cm and 20 mS/cm, 4 mS/cm and 10 mS/cm, 4 mS/cm and 7 mS/cm, 5 mS/cm and 17 mS/cm, 5 mS/cm and 10 mS/cm, or 5 mS/cm and 7 mS/cm. In some embodiments, the conductivity is about any of 1.0 mS/cm, 1.5 mS/cm, 2.0 mS/cm, 2.5 mS/cm, 3.0 mS/cm, 3.5 mS/cm, 4 mS/cm, 4.5 mS/cm, 5.0 mS/cm, 5.5 mS/cm, 6.0 mS/cm, 6.5 mS/cm, 7.0 mS/cm, 7.5 mS/cm, 8.0 mS/cm, 8.5 mS/cm, 9.0 mS/cm, 9.5 mS/cm, 10 mS/cm or 20 mS/cm. In certain embodiments, the conductivity is the conductivity of the loading buffer, the equilibration buffer, and/or the wash buffer. In certain embodiments, the conductivity of one or more of the loading buffer, the equilibration buffer, and the wash buffer is the same. In certain embodiments, the conductivity of the loading buffer is different from the conductivity of the wash buffer and/or equilibration buffer.

In certain embodiments, the elution buffer has a conductivity less than the conductivity of the loading buffer. In certain embodiments, the elution buffer has a conductivity of less than about any of 0 mS/cm, 0.5 mS/cm, 1.0 mS/cm, 1.5 mS/cm, 2.0 mS/cm, 2.5 mS/cm, 3.0 mS/cm, 3.5 mS/cm, 4.0 mS/cm, 4.5 mS/cm, 5.0 mS/cm, 5.5 mS/cm, 6.0 mS/cm, 6.5 mS/cm, or 7.0 mS/cm. In certain embodiments, the conductivity may be between about any of 0 mS/cm and 7 mS/cm, 1 mS/cm and 7 mS/cm, 2 mS/cm and 7 mS/cm, 3 mS/cm and 7 mS/cm, or 4 mS/cm and 7 mS/cm, 0 mS/cm and 5.0 mS/cm, 1 mS/cm and 5 mS/cm, 2 mS/cm and 5 mS/cm, 3 mS/cm and 5 mS/cm, or 4 mS/cm and 5 mS/cm. In certain embodiments, the conductivity of the elution buffer is about any of 0 mS/cm, 0.5 mS/cm, 1.0 mS/cm, 1.5 mS/cm, 2.0 mS/cm, 2.5 mS/cm, 3.0 mS/cm, 3.5 mS/cm, 4 mS/cm, 4.5 mS/cm, 5.0 mS/cm, 5.5 mS/cm, 6.0 mS/cm, 6.5 mS/cm, or 7.0 mS/cm.

In certain embodiments, the elution buffer has a conductivity greater than the conductivity of the loading buffer. In certain embodiments, the elution buffer has a conductivity of greater than about any of 5.5 mS/cm, 6.0 mS/cm, 6.5 mS/cm, 7.0 mS/cm, 7.5 mS/cm, 8.0 mS/cm, 8.5 mS/cm, 9.0 mS/cm, 9.5 mS/cm, 10 mS/cm, 11 mS/cm, 12 mS/cm, 13 mS/cm, 14 mS/cm, 15 mS/cm, 16 mS/cm, 17.0 mS/cm, 18.0 mS/cm, 19.0 mS/cm, 20.0 mS/cm, 21.0 mS/cm, 22.0 mS/cm, 23.0 mS/cm, 24.0 mS/cm, 25.0 mS/cm, 26.0 mS/cm, 27.0 mS/cm, 28.0 mS/cm, 29.0 mS/cm, or 30.0 mS/cm. In certain embodiments, the conductivity may be between about any of 5.5 mS/cm and 30 mS/cm, 6.0 mS/cm and 30 mS/cm, 7 mS/cm and 30 mS/cm, 8 mS/cm and 30 mS/cm, 9 mS/cm and 30 mS/cm, or 10 mS/cm and 30 mS/cm. In certain embodiments, the conductivity of the elution buffer is about any of 5.5 mS/cm, 6.0 mS/cm, 6.5 mS/cm, 7.0 mS/cm, 7.5 mS/cm, 8.0 mS/cm, 8.5 mS/cm, 9.0 mS/cm, 9.5 mS/cm, 10 mS/cm, 11 mS/cm, 12 mS/cm, 13 mS/cm, 14 mS/cm, 15 mS/cm, 16 mS/cm, 17.0 mS/cm 18.0 mS/cm, 19.0 mS/cm, 20.0 mS/cm, 21.0 mS/cm, 22.0 mS/cm, 23.0 mS/cm, 24.0 mS/cm, 25.0 mS/cm, 26.0 mS/cm, 27.0 mS/cm, 28.0 mS/cm, 29.0 mS/cm, or 30.0 mS/cm. In certain embodiments, the conductivity of the elution buffer is changed from the load and/or wash buffer by step gradient or by linear gradient.

In certain embodiments, the composition comprising the multispecific antibody is loaded onto the multi-mode chromatography material in a loading buffer with a conductivity of less than about 100 mS/cm and the polypeptide is eluted from the mixed chromatography material in an elution buffer with a conductivity of less than about 100 mS/cm. In certain embodiments, the loading buffer has a conductivity of less than about 100 mS/cm and the elution buffer has a conductivity of less than about 100 mS/cm. In certain embodiments, the loading buffer has a conductivity of less than about 100 mS/cm and the elution buffer has a conductivity of less than about 100 mS/cm. In certain embodiments, the loading buffer has a conductivity of less than about 100 mS/cm and the elution buffer has a conductivity of about xxx mS/cm. In certain embodiments, the multi-mode chromatography material is a Capto™ Adhere resin. In certain embodiments, the multi-mode chromatography material is a Capto™ MMC resin.

In certain embodiments, the conductivity of the elution buffer is changed from the load and/or wash buffer by step gradient or by linear gradient.

In certain embodiments, the loading buffer has a pH of less than about any of 10, 9, 8, 7, 6, or 5, including any range in between these values. In certain embodiments, the loading buffer has a pH of greater than about any of 4, 5, 6, 7, 8, or 9, including any range in between these values. In certain embodiments, the loading buffer can have a pH of between about any of 4 and 9, 4 and 8, 4 and 7, 5 and 9, 5 and 8, 5 and 7, 5 and 6, including any range in between these values. In certain embodiments, the pH of the loading buffer has a pH of about any of 4, 4.5, 5, 5.5, 6, 6.5, 7, 7.5, 8, or 8.5 including any range in between these values.

In certain embodiments, the elution has a pH less than the pH of the load buffer. In certain embodiments, the elution buffer has a pH of less than about any of 8, 7, 6, 5, 4, 3 or 2, including any range in between these values. In certain embodiments, the pH of the elution buffer may be between about any of 4 and 9, 4 and 8, 4 and 7, 4 and 6, 4 and 5, 5 and 9, 5 and 8, 5 and 7, 5 and 6, 6 and 9, 6 and 8, 6 and 7, including any range in between these values. In certain embodiments, the pH of the elution buffer is about any of 4.0, 4.5, 5.0, 5.5, 6.0, 6.5, 7.0, 7.5, 8.0, 8.5 or 9.0, including any range in between these values.

In certain embodiments, the elution buffer has a pH greater than the pH of the loading buffer. In certain embodiments, the elution buffer has a pH of greater than about any of 5, 6, 7, 8, or 9, including any range in between these values. In certain embodiments, the elution buffer has a pH of greater than about any of 2, 4, or 4, including any range in between these values. In certain embodiments, the pH of the elution buffer can be between about any of 2 and 9, 3 and 9, 4 and 9, 2 and 8, 3 and 8, 4 and 8, 2 and 7, 3 and 7, 4 and 7, 2 and 6, 3 and 6, and 4 and 6, including any range in between these values. In some embodiments, the pH of the elution buffer is about any of 2.0, 2.5, 3.0, 3.5, 4.0, including any range in between these values.

In certain embodiments, the solution comprising a multispecific antibody is loaded onto an affinity chromatography (e.g., a Protein A chromatography) at about pH 7 and the multispecific antibody or antibody arm is eluted from the affinity chromatography by a step gradient to pH of about 2.9.

In certain embodiments, the pH of the elution buffer is changed from the load and/or wash buffer by step gradient or by linear gradient.

In certain embodiments, the flow rate is less than about any of 50 CV/hr, 40 CV/hr, or 30 CV/hr. The flow rate may be between about any of 5 CV/hr and 50 CV/hr, 10 CV/hr and 40 CV/hr, or 18 CV/hr and 36 CV/hr. In certain embodiments, the flow rate is about any of 9 CV/hr, 18 CV/hr, 25 CV/hr, 30 CV/hr, 36 CV/hr, or 40 CV/hr. In certain embodiments, the flow rate is less than about any of 100 cm/hr, 75 cm/hr, or 50 cm/hr. In certain embodiments, the flow rate can be between about any of 25 cm/hr and 150 cm/hr, 25 cm/hr and 100 cm/hr, 50 cm/hr and 100 cm/hr, or 65 cm/hr and 85 cm/hr.

Bed height is the height of chromatography material used. In certain embodiments, the bed height is greater than about any of 5 cm, 10 cm, 15 cm, 20 cm, 25 cm, 30 cm, 35 cm, 40 cm, 45 cm, or 50 cm. In certain embodiments, the bed height is between about 5 cm and 50 cm. In certain embodiments, bed height is determined based on the amount of polypeptide or contaminants in the load.

In certain embodiments, the chromatography is in a column or vessel with a volume of greater than about 1 mL, 2 mL, 3 mL, 4 mL, 5 mL, 6 mL, 7 mL, 8 mL, 9 mL, 10 mL, 15 mL, 20 mL, 25 mL, 30 mL, 40 mL, 50 mL, 75 mL, 100 mL, 200 mL, 300 mL, 400 mL, 500 mL, 600 mL, 700 mL, 800 mL, 900 mL, 1 L, 2 L, 3 L, 4 L, 5 L, 6 L, 7 L, 8 L, 9 L, 10 L, 25 L, 50 L, 100 L, 200 L, 300 L, 400 L, 500 L, 600 L, 700 L, 800 L, 900 L or 1000 L.

In certain embodiments, fractions are collected from the chromatography. In certain embodiments, fractions collected are greater than about 0.01 CV, 0.02 CV, 0.03 CV, 0.04 CV, 0.05 CV, 0.06 CV, 0.07 CV, 0.08 CV, 0.09 CV, 0.1 CV, 0.2 CV, 0.3 CV, 0.4 CV, 0.5 CV, 0.6 CV, 0.7 CV, 0.8 CV, 0.9 CV, 1.0 CV, 2.0 CV, 3.0 CV, 4.0 CV, 5.0 CV, 6.0 CV, 7.0 CV, 8.0 CV, 9.0 CV, or 10.0 CV.

In certain embodiments, fractions containing the purified product, e.g., the multispecific antibody (such as a bispecific antibody), are pooled. In certain non-limiting embodiments, the amount of polypeptide in a fraction can be determined by one skilled in the art. For example, but without any limitation, the amount of polypeptide in a fraction can be determined by UV spectroscopy. In certain embodiments, fractions are collected when the OD280 is greater than about any of 0.5, 0.6, 0.7, 0.8, 0.9 and 1.0. In certain embodiments, fractions are collected when the OD280 is between about any of 0.5 and 1.0, 0.6 and 1.0, 0.7 and 1.0, 0.8 and 1.0, or 0.9 and 1.0. In certain embodiments, fractions containing detectable multispecific antibody (e.g., bispecific antibody) are pooled.

In certain embodiments, the impurity is a product specific impurity. For example, without any limitation, product specific impurities include unpaired half-antibody, un-paired antibody light chains, unpaired heavy chains, mispaired antibodies, antibody fragments, homodimers (e.g., paired half-dimers of a bispecific antibody that comprise the same heavy and light chain), aggregates, high molecular weight species (MHWS) (such as very high molecular weight species (vHMWS)), multispecific antibodies with mispaired disulfides, light chain dimers, heavy chain dimers, low molecular weight species (LMWS), and other variants. FIGS. 2A and 2B illustrate graphical examples of product specific impurities.

In certain embodiments, the present disclosure provides methods for removing or reducing the level of light-chain mispaired multispecific antibody from a composition comprising a multispecific antibody (e.g., a bispecific antibody) and impurities. In certain embodiments, the present disclosure provides methods of measuring the presence or level of light-chain mispaired antibody in a composition. For example, but without any limitation, light-chain mispaired antibody can be measured by mass spectrometry, CE-SDS, Reverse Phase HPLC, HIC HPLC. In certain embodiments, the amount of light-chain mispaired antibody in a composition recovered from one or more purification step(s) is reduced by more than about any of 5%, 10%, 15%, 20%, 25%, 30%, 35%, 40%, 45%, 50%, 55%, 60%, 65%, 70%, 75%, 80%, 85%, 90%, 95%, or 99%, including any range in between these values. In certain embodiments, the amount of light-chain mispaired antibody in a composition recovered from one or more purification step(s) is reduced by between about any of 10 and 95%; 10% and 99%; 20% and 95%; 20% and 99%; 30% and 95%; 30% and 99%; 40% and 95%; 40% and 99%; 50% and 95%; 50% and 99%; 60% and 95%; 60% and 99%; 70% and 95%; 70% and 99%; 80% and 95%; 80% and 99%; 90% and 95%; or 90% and 99%.

In certain embodiments, the multispecific antibody is concentrated after chromatography (e.g., after the multi-mode chromatography). In certain non-limiting embodiments, for example, concentration methods include ultrafiltration and diafiltration (UFDF). In certain embodiments, the concentration of multispecific antibody following concentration is about any of 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 110 mg/mL, 120 mg/mL, 130 mg/mL, 140 mg/mL, 150 mg/mL, 160 mg/mL, 170 mg/mL, 180 mg/mL, 190 mg/mL, 200 mg/mL, or 300 mg/mL. In certain embodiments, the concentration of multispecific antibody is between about any of 10 mg/mL and 20 mg/mL, 20 mg/mL and 30 mg/mL, 30 mg/mL and 40 mg/mL, 40 mg/mL and 50 mg/mL, 50 mg/mL and 60 mg/mL, 60 mg/mL and 70 mg/mL, 70 mg/mL and 80 mg/mL, 80 mg/mL and 90 mg/mL, 90 mg/mL and 100 mg/mL, 100 mg/mL and 110 mg/mL, 110 mg/mL and 120 mg/mL, 120 mg/mL and 130 mg/mL, 130 mg/mL and 140 mg/mL, 140 mg/mL and 150 mg/mL, 150 mg/mL and 160 mg/mL, 160 mg/mL and 170 mg/mL, 170 mg/mL and 180 mg/mL, 180 mg/mL and 190 mg/mL, 190 mg/mL and 200 mg/mL, 200 mg/mL or 300 mg/mL.

In certain embodiments, the methods described herein further comprise combining the purified polypeptide with a pharmaceutically acceptable carrier. In certain embodiments, the multispecific antibody is formulated into a pharmaceutical formulation by ultrafiltration/diafiltration.

In certain embodiments, the methods provided herein produce a composition comprising a multispecific antibody that is more than about any of 50%, 55%, 60%, 65%, 70%, 75%, 80%, 85%, 90%, 95% pure. In certain embodiments, the multispecific antibody in the composition is more than about any of 96%, 97%, 98%, or 99% pure.

In certain embodiments, the methods provided herein produce a composition comprising the multispecific antibody contains no more than about any of 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% mispaired antibody.

In certain embodiments, the present disclosure provides a composition comprising a multispecific antibody purified according to any one of the methods disclosed herein. In certain embodiments, the multispecific antibody in the composition is more than about any of 50%, 55%, 60%, 65%, 70%, 75%, 80%, 85%, 90%, 95% pure. In certain embodiments, the multispecific antibody in the composition is more than about any of 96%, 97%, 98%, or 99% pure. In certain embodiments, the composition comprising the multispecific antibody contains no more than about any of 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% mispaired antibody.

In certain embodiments, the present disclosure provides a composition comprising a multispecific antibody purified according to any one of the methods disclosed herein. In certain embodiments, the multispecific antibody is a bispecific antibody is a knob-in-hole (KiH) antibody, e.g., a KiH bispecific antibody. In certain embodiments, the multispecific antibody is a multispecific CrossMab antibody, e.g., a bispecific CrossMab antibody.

In certain embodiments, the methods disclosed herein comprise the removal of host cell proteins, leached protein A, nucleic acids, cell culture media components, or viral impurities in the composition

Multispecific Antibodies

In certain non-limiting embodiments, the present disclosure provides methods for purifying a multispecific antibody, e.g. a multispecific CrossMab antibody. Multispecific antibodies are monoclonal antibodies that have binding specificities for at least two different sites. In certain embodiments, the multispecific antibodies are produced by the same host cell.

In certain embodiments, the present disclosure comprises methods for making multispecific antibodies. For example, but without any limitation, these techniques compriserecombinant co-expression of two immunoglobulin heavy chain-light chain pairs having different specificities (see Milstein and Cuello, Nature 305: 537 (1983), WO 93/08829, and Traunecker et al., EMBO J. 10: 3655 (1991)), “knob-in-hole” engineering (see, e.g., U.S. Pat. No. 5,731,168), and “CrossMab” antibodies (see, e.g., European Patent No. EP3126395B1). Multi-specific antibodies can be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies (see, e.g., Tutt et al., J. Immunol. 147: 60 (1991)).

In certain embodiments, the multispecific antibodies are described in WO 2009/080251, WO 2009/080252, WO 2009/080253, WO 2009/080254, WO 2010/112193, WO 2010/115589, WO 2010/136172, WO 2010/145792, and WO 2010/145793. In certain embodiments, the multispecific antibodies comprise three or more functional antigen binding sites such as “Octopus antibodies,” (see, e.g. US 2006/0025576A1). In certain embodiment, the multispecific antibody is a “Dual Acting Fab” or “Dual Action Fab” (DAF) comprising an antigen binding site that binds to a first epitope (e.g., on a first antigen) as well as another, different epitope (e.g., on the first antigen or on a second, different antigen) (see, e.g., US 2008/0069820; Bostrom et al. (2009) Science, 5921, 1610-1614).

Traditionally, the recombinant production of multispecific antibodies (e.g., bispecific antibodies) can be based on the co-expression of two immunoglobulin heavy chain-light chain pairs, where the two or more heavy chains have different specificities (Milstein and Cuello, Nature, 305: 537 (1983)). Because of the random assortment of immunoglobulin heavy and light chains, these hybridomas (quadromas) produce a potential mixture of at least 10 different antibody molecules, of which only one has the correct bispecific structure. The purification of the correct molecule, which is usually done by affinity chromatography steps, is rather cumbersome, and the product yields are low. Similar procedures are disclosed in WO 93/08829 published May 13, 1993, and in Traunecker et al., EMBO J., 10: 3655 (1991).

In addition, the production of multispecific antibodies presents specific challenges. For example, the production of a bispecific antibody requires the dimerization of two different heavy-chain/light-chain subunits, each comprising a different heavy chain as well as a different light chain. Thus, bispecific antibody production requires the proper interaction of up to four peptide chains. Accordingly, chain mispairings (e.g., homo-dimerization of identical heavy chain peptides or improper heavy-chain/light-chain associations) are often observed. The mispaired variants of a multispecific antibody comprise the pairing of wrong heavy chains with each other as well as pairing of a light chain with a wrong heavy chain counterpart or undesired pairing of light chains.

CrossMab Antibody

The present disclosure provides methods for purifying a multispecific CrossMab antibody. CrossMab antibodies are multispecific (i.e. at least bispecific) antibodies in which correct association of the light chains and their cognate heavy chains is achieved by exchange of heavy-chain and light-chain domains within the antigen binding region (Fab) of at least one Fab of the multispecific antibody wherein no such exchange is performed in at least one other Fab fragment so that mispairing is avoided in these at least two Fab fragments. In the case of bispecific CrossMab antibodies, correct association of the light chains and their cognate heavy chains can, thus, be achieved by exchange of heavy-chain and light-chain domains within the Fab fragment of one half of the bispecific antibody while the other half remains unchained or has a different exchange.

As used herein, the term “CrossMab antibody” refers to a multispecific antibody (or a suitable multispecific fragment thereof), wherein either the variable regions and/or the constant regions of the heavy and light chain are exchanged. For example, the CrossMab antibody can be any of the CrossMab antibodies described or claimed in WO 2009/080252, WO 2009/080253, WO 2009/080251, WO 2009/080254, WO 2010/136172, WO 2010/145792 and WO 2013/026831. The term “CrossMab” antibody is generally recognized in the art; e.g. see Brinkmann, U. & Kontennann, R., MAbs 9(2): 182-212 (2017); Kontermann, R. & Brinkmann, U., Drug Discovery Today 20(7):838-846 (2015); Schaefer, W. et a, PNAS, 108 (201 1) 11187-1 191; Klein, C. et al., MAbs 8(6):1010-1020 (2016); Klein, C. et al., MAbs 4(6):653-663 (2012).

In certain embodiments, the multispecific CrossMab antibody is a bispecific bivalent CrossMab antibody. A bispecific bivalent CrossMab antibody comprises three different chain compositions of a crossover antibody. In the first composition, the variable domains of the heavy and light chain of the antibody are exchanged, i.e. the antibody comprises in one Fab region a peptide chain composed of the light chain variable domain (VL) and the heavy chain constant domain (CH1), and a peptide chain composed of the heavy chain variable domain (VH) and the light chain constant domain (CL). In the second composition, the constant domains of the heavy and light chain of the antibody in one Fab region are exchanged and the antibody comprises in this Fab region a peptide chain composed of the heavy chain variable domain (VH) and the light chain constant domain (CL), and a peptide chain composed of the light chain variable domain (VL) and the heavy chain constant domain (CH1). In the third composition, the heavy chain of the antibody comprising the constant and the variable domains and the light chain of the antibody comprising the constant and the variable domain are exchanged, i.e. the antibody comprises a peptide chain composed of the light chain variable domain (VH) and the heavy chain constant domain (VL), and a peptide chain composed of the heavy chain variable domain (VL) and the light chain constant domain (CH1).

In certain embodiments, CrossMab antibodies are monoclonal antibodies. In certain embodiments, CrossMab antibodies comprise functional fragments thereof, i.e. fragment that retain their multispecificity.

In certain embodiments, the present disclosure provides methods for purifying a multispecific CrossMab antibody from mispaired variants thereof. As used herein, the term “mispaired variant thereof” refers to a multispecific CrossMab antibody that is paired with at least one wrong light chain with the domain-exchanged heavy chain as described above with respect to the CrossMab antibody. For example, but without any limitation, at least one of the light chains of said variant does not pair with its complementary heavy chain, e.g. an “unmodified” light chain comprising CL and VL mispairs with a “modified” heavy chain having CH1 and VL or a “modified” light chain comprising CH1 and VL mispairs with an “unmodified” heavy chain having CH1 and VH etc. As used in reference to CrossMab antibody, the “complementary” domains are the normally pairing heavy and light chain domains. Alternatively, the “non-complementary” domains are the wrong pairing heavy and light chain domains. For example, without any limitation, the wrong light chain of the pair of heavy and light chain domains may refer to a light chain, wherein the variable and/or constant domains of the light chain are exchanged, whereas in the heavy chain the variable and/or constant domains of the heavy chain are not exchanged. As another example, the wrong pairing of heavy and light chain domains may refer to a situation in which the variable and/or constant domains of the light chain are not exchanged, and the variable and/or constant domains of the heavy chain are exchanged. As used herein, the term “non-complementary” does not refer to incompletely assembled antibodies, such as but not limited to antibodies in which one light chain or a fragment thereof is missing. In certain non-limiting embodiments, for example, the mispaired variant thereof is a variant of the multispecific CrossMab antibody, wherein one or more light chains are paired with a non-complementary heavy chain.

In certain embodiments, the multispecific CrossMab antibody is a bispecific, trispecific, or tetraspecific antibody. In certain embodiments, the multispecific CrossMab antibody has two, three, or four specific antigen binding sites. In certain embodiments, the multispecific CrossMab antibody is monovalent. In certain embodiments, the multispecific CrossMab antibody is bivalent.

In certain embodiments, the multispecific CrossMab antibody comprises an Fc fragment. The presence of an Fc fragment allows the multispecific antibody to be purified by using Fc-binding moieties such as, without any limitation, Protein A, Protein G, or Protein A/G. In certain embodiments, the multispecific CrossMab antibody can be IgG, IgE, IgM, IgA, or IgY. In certain embodiments, the multispecific CrossMab antibody is an IgG. In certain embodiments, the Fc fragment of the multispecific antibody comprises a modification promoting the association of a first and a second Fc fragment subunit. In certain embodiments, the modification is in the first Fc fragment subunit. In certain embodiments, the modification is in the second Fc fragment subunit. In certain embodiments, the modification is in the first and second Fc fragment subunits. In certain embodiments, the modification in in the CH3 domain of the Fc fragment. In certain non-limiting embodiments, the modification of the first and second CH3 domains allows the correct heterodimerization of the Fc fragments. In certain embodiments, the modified first CH3 domain heterodimerize with the modified second CH3 domain by steric complementarity.

In certain embodiments, the modification is a “knob-into-hole” modification. In certain embodiments, the first Fc fragment comprises a knob mutation and the second Fc fragment comprises a hole mutation. In certain embodiments, the first Fc fragment comprises a hole mutation and the second Fc fragment comprises a knob mutation.

Host Cells

The present disclosure provides methods for purifying a multispecific antibody expressed in a host cell. In certain embodiments, the host cell is a bacterium, a yeast or other fungal cell, insect cell, a plant cell, or a mammalian cell. In certain embodiments, the host cell has been genetically modified to produce the multispecific antibody.

In certain embodiments, the host cell is a prokaryote (e.g., a Gram-negative or Gram-positive organism). For example, but without any limitation, the host cell is E. coli, B. subtilis, B. licheniformis, or P. aeruginosa. In certain embodiments, the host cell secretes minimal amounts of proteolytic enzymes. In certain embodiments, the host cell (e.g., an E. coli host cell) expresses one or more chaperones to facilitate folding and assembly of the antibody. In certain embodiments, the chaperone is one or more of FkpA, DsbA or DsbC. In certain embodiments, the chaperone is expressed from an endogenous chaperone gene. In certain embodiments, the chaperone is expressed from an exogenous chaperone gene. In certain embodiments, the chaperone gene is an E. coli chaperone gene (e.g., an E. coli FkpA gene, an E. coli DsbA gene and/or an E. coli DsbC gene).

In certain embodiments, the prokaryote host cell is transformed with an expression vector and is cultured to promote the expression of the multispecific antibody.

In certain embodiments, the host cell is a eukaryote. For example, but without any limitation, the host cell is Saccharomyces cerevisiae, Pichia pastoris, Neurospora crassa, or A. niger. In certain embodiments, the eukaryotic host cell is a mammalian cell. In certain non-limiting embodiments, the mammalian host cell is a CHO cell, a COS-7 cell, a HEK 293 cell, a BHK cell, a VERO-76 cell, a HELA cell, a HepG2 cell, or a W138 cell. In certain embodiments, the eukaryote host cell is transformed with an expression vector and is cultured to promote the expression of the multispecific antibody. In certain non-limiting embodiments, the present disclosure provides methods for producing and purifying multispecific antibodies. In certain embodiments, multispecific antibodies are produced by separately producing half-antibodies, each half antibody comprising a VH/VL unit that binds a specific epitope (e.g., different epitopes on a single target, or different epitopes on two or more targets). In certain embodiments, each half-antibody is produced separately in a host cell. In certain embodiments, each of the half-antibodies is produced in the same host cell. In certain embodiments, each of the half-antibodies is produced together in the same host cell.

Antigens/Target Molecules

The present disclosure provides method for purifying multispecific antibodies capable of targeting various molecules. In certain embodiments, the multispecific antibodies purified according to methods disclosed herein can target a cytokine, a cytokine-related protein, or a cytokine receptor. For example, but without any limitation, the multispecific antibody can target 8MPI, 8MP2, 8MP38 (GDFIO), 8MP4, 8MP6, 8MP8, CSFI (M-CSF), CSF2 (GM-CSF), CSF3 (G-CSF), EPO, FGF1 (aFGF), FGF2 ((FGF), FGF3 (int-2), FGF4 (HST), FGFS, FGF6 (HST-2), FGF7 (KGF), FGF9, FGF1 0, FGF11, FGF12, FGF12B, FGF14, FGF16, FGF17, FGF19, FGF20, FGF21, FGF23, IGF1, IGF2, IFNA1, IFNA2, IFNA4, IFNA5, IFNA6, IFNA7, IFN81, IFNG, IFNWI, FEL1, FEL1 (EPSELON), FEL1 (ZETA), IL1A, IL1B, IL2, IL3, IL4, IL5, IL6, IL7, IL8, IL9, IL1 0, IL 11, IL 12A, IL 12B, IL 13, IL 14, IL 15, IL 16, IL 17, IL 17B, IL 18, IL 19, IL20, IL22, IL23, IL24, IL25, IL26, IL27, IL28A, IL28B, IL29, IL30, IL33, PDGFA, PDGFB, TGFA, TGFB1, TGFB2, TGFBb3, LTA (TNF-( ), LTB, TNF (TNF-α), TNFSF4 (0X40 ligand), TNFSF5 (CD40 ligand), TNFSF6 (FasL), TNFSF7 (CD27 ligand), TNFSF8 (CD30 ligand), TNFSF9 (4-1 BB ligand), TNFSF10 (TRAIL), TNFSF11 (TRANCE), TNFSF12 (APO3L), TNFSF13 (April), TNFSF13B, TNFSF14 (HVEM-L), TNFSF15 (VEGI), TNFSF18, HGF (VEGFD), VEGF, VEGFB, VEGFC, IL1R1, IL1R2, IL1RL1, IL1RL2, IL2RA, IL2RB, IL2RG, IL3RA, IL4R, IL5RA, IL6R, IL7R, IL8RA, IL8RB, IL9R, IL10RA, IL10RB, IL 11RA, IL12RB1, IL12RB2, IL13RA1, IL13RA2, IL15RA, IL17R, IL18R1, IL20RA, IL21R, IL22R, IL1HY1, IL1RAP, IL1RAPL1, IL1RAPL2, IL1RN, IL6ST, IL18BP, IL18RAP, IL22RA2, AIF1, HGF, LEP (leptin), PTN, and THPO.

In certain embodiments, the multispecific antibodies purified according to methods disclosed herein can target a chemokine, a chemokine receptor, or a chemokine-related protein. For example, but without any limitation, the multispecific antibody can target CCL1 (1-309), CCL2 (MCP-1/MCAF), CCL3 (MIP-1a), CCL4 (MIP-1(3), CCLS (RANTES), CCL7 (MCP-3), CCL8 (mcp-2), CCL11 (eotaxin), CCL13 (MCP-4), CCL15 (MIP-IS), CCL16 (HCC-4), CCL17 (TARC), CCL18 (PARC), CCL19 (MDP-3b), CCL20 (MIP-3a), CCL21 (SLC/exodus-2), CCL22 (MDC/STC-1), CCL23 (MPIF-1), CCL24 (MPIF-2/eotaxin-2), CCL25 (TECK), CCL26 (eotaxin-3), CCL2? (CTACK/ILC), CCL28, CXCLI (GROI), CXCL2 (GRO2), CXCL3 (GRO3), CXCLS (ENA-78), CXCL6 (GCP-2), CXCL9 (MIG), CXCL10 (IP 10), CXCL11 (1-TAC), CXCL12 (SDFI), CXCL13, CXCL14, CXCL16, PF4 (CXCL4), PPBP (CXCL7), CX3CL1 (SCYDI), SCYEI, XCLI (lymphotactin), XCL2 (SCM-I(3), BLRI (MDR15), CCBP2 (D6/JAB61), CCR1 (CKRI/HM145), CCR2 (mcp-IRB IRA), CCR3 (CKR3/CMKBR3), CCR4, CCRS (CMKBR5/ChemR13), CCR6 (CMKBR6/CKR-L3/STRL22/DRY6), CCRI (CKR7/EBII), CCR8 (CMKBR8/TERUCKR-L1), CCR9 (GPR-9-6), CCRL1 (VSHK1), CCRL2 (L-CCR), XCR1 (GPRS/CCXCR1), CMKLR1, CMKOR1 (RDC1), CX3CR1 (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Ra), IL8RB (IL8R( ), LTB4R (GPR16), TCP10, CKLFSF2, CKLFSF3, CKLFSF4, CKLFSFS, CKLFSF6, CKLFSF7, CKLFSF8, BDNF, C5R1, CSF3, GRCC10 (C10), EPO, FY (DARC), GDFS, HDF1, HDFla, DL8, PRL, RGS3, RGS13, SDF2, SLIT2, TLR2, TLR4, TREM1, TREM2, and VHL.

In certain non-limiting embodiments, for example, the multispecific antibodies purified according to methods disclosed herein can target ABCF1, ACVR1, ACVR1B, ACVR2, ACVR2B, ACVRL1, ADORA2A, Aggrecan, AGR2, AICDA, AIF1, AIG1, AKAP1, AKAP2, AMH, AMHR2, ANGPTL, ANGPT2, ANGPTL3, ANGPTL4, ANPEP, APC, APOC1, AR, AZGP1 (zinc-a-glycoprotein), B7.1, B7.2, BAD, BAFF (BLys), BAG1, BAIL BCL2, BCL6, BDNF, BLNK, BLRI (MDR15), BMP1, BMP2, BMP3B (GDF10), BMP4, BMP6, BMP8, BMPR1A, BMPR1B, BMPR2, BPAG1 (plectin), BRCA1, Cl9orf10 (IL27w), C3, C4A, C5, C5R1, CA125, CA15-3, CA19-9, CANT1, CASP1, CASP4, CAV1, CCBP2 (D6/JAB61), CCL1 (1-309), CCL11 (eotaxin), CCL13 (MCP-4), CCL15 (MIP18), CCL16 (HCC-4), CCL17 (TARC), CCL18 (PARC), CCL19 (MIP-3(3), CCL2 (MCP-1), MCAF, CCL20 (MIP-3a), CCL21 (MTP-2), SLC, exodus-2, CCL22 (MDC/STC-1), CCL23 (MPIF-1), CCL24 (MPIF-2/eotaxin-2), CCL25 (TECK), CCL26 (eotaxin-3), CCL2? (CTACK/ILC), CCL28, CCL3 (MTP-Ia), CCL4 (MDP-I(3), CCLS(RANTES), CCL7 (MCP-3), CCL8 (mcp-2), CCNA1, CCNA2, CCND1, CCNE1, CCNE2, CCR1 (CKRI/HM145), CCR2 (mcp-IR(3/RA),CCR3 (CKR/CMKBR3), CCR4, CCR5 (CMKBR5/ChemR13), CCR6 (CMKBR6/CKR-L3/STRL22/DRY6), CCR7 (CKBR7/EBI1), CCR8 (CMKBR8/TERUCKR-L1), CCR9 (GPR-9-6), CCRL1 (VSHK1), CCRL2 (L-CCR), CD11a, CD13, CD164, CD19, CD1C, CD20, CD200, CD22, CD23, CD24, CD28, CD3, CD30, CD31, CD33, CD34, CD35, CD37, CD38, CD39, CD3E, CD3G, CD3Z, CD4, CD40, CD40L, CD41, CD44, LCA/CD45, CD45RA, CD45RB, CD45RO, CDS, CD52, CD69, CD7, CD71, CD72, CD74, CD79A, CD79B, CD8, CD80, CD81, CD83, CD86, CD95/Fas, CD99, CD100, CD106, CDH1 (E-cadherin), CD9/p24, CDH10, CD11a, CD11c, CD13, CD14, CD19, CD20, CDH12, CDH13, CDH18, CDH19, CDH2O, CDH5, CDH7, CDH8, CDH9, CDK2, CDK3, CDK4, CDK5, CDK6, CDK7, CDK9, CDKN1A (p21/WAF1/Cipl), CDKN1B (p27/Kipl), CDKN1C, CDKN2A (P16INK4a), CDKN2B, CDKN2C, CDKN3, CEA, CEBPB, CER1, CHGA, CHGB, Chitinase, CHST10, CKLFSF2, CKLFSF3, CKLFSF4, CKLFSFS, CKLFSF6, CKLFSF7, CKLFSF8, CLDN3, CLDN7 (claudin-7), CLN3, CLU (clusterin), C-MET, CMKLR1, CMKOR1 (RDC1), CNR1, COL 18A1, COL1A1, COL4A3, COL6A1, CR2, CRP, CSFI (M-CSF), CSF2 (GM-CSF), CSF3 (GCSF), CTLA4, CTNNB1 (b-catenin), CTSB (cathepsin B), CTSD (cathepsin D), CX3CL1 (SCYDI), CX3CR1 (V28), CXCL1 (GRO1), CXCL10 (IP-10), CXCL11 (I-TAC/IP-9), CXCL12 (SDF1), CXCL13, CXCL14, CXCL16, CXCL2 (GRO2), CXCL3 (GRO3), CXCLS (ENA-78/LIX), CXCL6 (GCP-2), CXCL9 (MIG), CXCR3 (GPR9/CKR-L2), CXCR4, CXCR6 (TYMSTR/STRL33/Bonzo), CYB5, CYCl, CYSLTR1, cytokeratins, DAB2IP, DES, DKFZp451J0118, DNCLI, DPP4, E2F1, ECGF1, EDG1, EFNA1, EFNA3, EFNB2, EGF, EGFR, ELAC2, ENG, ENO1, ENO2, ENO3, EPHB4, EPO, ERBB2 (Her-2), EREG, ERK8, ESR1, estrogen receptor, progesterone receptor, ESR2, F3 (TF), FADD, FasL, FASN, FCER1A, FCER2, FCGR3A, FGF, FGF1 (aFGF), FGF10, FGF11, FGF12, FGF12B, FGF13, FGF14, FGF16, FGF17, FGF18, FGF19, FGF2 (bFGF), FGF20, FGF21, FGF22, FGF23, FGF3 (int-2), FGF4 (HST), FGF5, FGF6 (HST-2), FGF7 (KGF), FGF8, FGF9, FGFR1, FGFR3, FIGF (VEGFD), FELL (EPSILON), fibrin, FIL1 (ZETA), FLJ12584, FLJ25530, FLRTI (fibronectin), FLT1, FOS, FOSL1 (FRA-1), FY (DARC), GABRP (GABAa), GAGEB1, GAGEC1, GALNAC4S-6ST, GATA3, GDF5, GFIl, GGT1, GM-CSF, GNASI, GNRHI, GPR2 (CCR10), GPR31, GPR44, GPR81 (FKSG80), GRCCIO (C10), GRP, GSN (Gelsolin), GSTP1, HAVCR2, HDAC4, HDAC5, HDAC7A, HDAC9, HGF, HIF1A, HOPI, histamine and histamine receptors, HLA-A, HLA-DRA, HM74, HMOXI, HPV proteins, HUMCYT2A, ICEBERG, ICOSL, 1D2, IFN-α, IFNA1, IFNA2, IFNA4, IFNA5, IFNA6, IFNA7, IFNB1, IFNgamma, ITGB7, DFNW1, IGBP1, IGF1, IGF1R, IGF2, IGFBP2, IGFBP3, IGFBP6, IL-1, IL1O, IL1ORA, IL1ORB, IL11, IL11RA, IL-12, IL12A, IL12B, IL12RB1, IL12RB2, IL13, IL13RA1, IL13RA2, IL14, IL15, IL15RA, IL16, IL17, IL17B, IL17C, IL17R, IL18, IL18BP, IL18R1, IL18RAP, IL19, ILIA, IL1B, IL1F1O, IL1F5, IL1F6, IL1F7, IL1F8, IL1F9, IL1HY1, IL1R1, IL1R2, IL1RAP, IL1RAPL1, IL1RAPL2, IL1RL1, IL1RL2, ILIRN, IL2, IL20, IL20RA, IL21 R, IL22, IL22R, IL22RA2, IL23, IL24, IL25, IL26, IL27, IL28A, IL28B, IL29, IL2RA, IL2RB, IL2RG, IL3, IL30, IL3RA, IL33, IL4, IL4R, IL5, IL5RA, IL6, IL6R, IL6ST (glycoprotein 130), P-glycoprotein, EL7, EL7R, EL8, IL8RA, DL8RB, IL8RB, DL9, DL9R, DLK, INHA, INHBA, INSL3, INSL4, IRAK1, ERAK2, ITGA1, ITGA2, ITGA3, ITGA6 (a6 integrin), ITGAV, ITGB3, ITGB4 (b4 integrin), JAG1, JAK1, JAK3, JUN, K6HF, KAII, KDR, keratin, KITLG, KLF5 (GC Box BP), KLF6, KLKIO, KLK12, KLK13, KLK14, KLK15, KLK3, KLK4, KLK5, KLK6, KLK9, KRT1, KRT19 (Keratin 19), KRT2A, KHTHB6 (hair-specific type H keratin), kappa light chain, lambda light chain, LAMAS, LEP (leptin), Lingo-p75, Lingo-Troy, LPS, LTA (TNF-b), LTB, LTB4R (GPR16), LTB4R2, LTBR, LEWIS-xMACMARCKS, MAG or Omgp, MAP2K7 (c-Jun), MDK, MD31, melanosome proteins, midkine, MEF, MIP-2, MKI67, (Ki-67), MMP2, MMP9, MS4A1, MSMB, MT3 (metallothionectin-111), MTSS1, MUC1 (mucin), MYC, MY088, NCK2, neurocan, NFKB1, NFKB2, NGFB (NGF), NGFR, NgR-Lingo, NgR-Nogo66 (Nogo), NgR-p75, NgR-Troy, NME1 (NM23A), NOX5, NPPB, NR0B1, NROB2, NR1D1, NR1D2, NR1H2, NR1H3, NR1H4, NR112, NR113, NR2C1, NR2C2, NR2E1, NR2E3, NR2F1, NR2F2, NR2F6, NR3C1, NR3C2, NR4A1, NR4A2, NR4A3, NR5A1, NR5A2, NR6A1, NRP1, NRP2, NT5E, NTN4, ODZI, OPRD1, P2RX7, PAP, PART1, PATE, PAWR, PCA3, PCNA, POGFA, POGFB, PECAM1, PF4 (CXCL4), PGF, PGR, phosphacan, PIAS2, PIK3CG, PLAU (uPA), PLG, PLXDC1, PPBP (CXCL7), PPID, PRI, PRKCQ, PRKDI, PRL, PROC, PROK2, PSA, PSAP, PSCA, PTAFR, PTEN, PTGS2 (COX-2), PTN, p53, RAC2 (p21 Rac2), RAS, Rb, RARE, RGSI, RGS13, RGS3, RNF110 (ZNF144), ROBO2, S100A2, SCGB1D2 (lipophilin B), SCGB2A1 (mammaglobin2), SCGB2A2 (mammaglobin 1), SCYEI (endothelial Monocyte-activating cytokine), S-100 SDF2, SERPINA1, SERPINA3, SERP1NB5 (maspin), SERPINE1(PAI-1), SERPDMF1, SHBG, SLA2, SLC2A2, SLC33A1, SLC43A1, SLIT2, SPPI, SPRR1B (Sprl), ST6GAL1, STABI, STATE, STEAP, STEAP2, TB4R2, TBX21, TCPIO, TOGFI, TEK, TGFA, TGFBI, a transmembrane or cell surface tumor specific antigen (TAA) such as a TAA described in USP 7,521, 541, TAU, TGFB1II, TGFB2, TGFB3, TGFBI, TGFBRI, TGFBR2, TGFBR3, THIL, THBSI (thrombospondin-1), THBS2, THBS4, THPO, TIE (Tie-1), TMP3, tissue factor, TLR1, TLR2, TLR3, TLR4, TLR5, TLR6, TLR7, TLR8, TLR9, TLR10, Tn antigen TNF, TNF-α, TNFAEP2 (B94), TNFAIP3, TNFRSFIIA, TNFRSF1A, TNFRSF1B, TNFRSF21, TNFRSFS, TNFRSF6 (Fas), TNFRSF7, TNFRSF8, TNFRSF9, TNFSF10 (TRAIL), TNFSF11 (TRANCE), TNFSF12 (APO3L), TNFSF13 (April), TNFSF13B, TNFSF14 (HVEM-L), TNFSF15 (VEGI), TNFSF18, TNFSF4 (0X40 ligand), TNFSF5 (CD40 ligand), TNFSF6 (Fast), TNFSF7 (CD27 ligand), TNFSFS (CD30 ligand), TNFSF9 (4-1 BB ligand), TOLLIP, Toll-like receptors, TOP2A (topoisomerase Ea), TP53, TPM1, TPM2, TRADD, TRAF1, TRAF2, TRAF3, TRAF4, TRAFS, TRAF6, TREM1, TREM2, TRPC6, TSLP, TWEAK, ubiquitin, VEGF, VEGFB, VEGFC, versican, VHL C5, vimentins, VLA-4, XCL1 (lymphotactin), XCL2 (SCM-1b), XCRI(GPRS/CCXCRI), YY1, and ZFPM2.

In certain non-limiting embodiments, for example, the multispecific antibodies purified according to methods disclosed herein can target CD proteins such as CD3, CD4, CD8, CD16, CD19, CD20, CD34, CD64, CD200 members of the ErbB receptor family such as the EGF receptor, HER2, HER3 or HER4 receptor, cell adhesion molecules such as LFA-1, Mad, p150.95, VLA-4, ICAM-1, VCAM, alpha4/beta7 integrin, and alphav/beta3 integrin including either alpha or beta subunits thereof (e.g., anti-CD11 a, anti-CD18, or anti-CD11b antibodies), growth factors such as VEGF (VEGF-A), FGFR, Angl, KLB, VEGF-C, tissue factor (TF), alpha interferon (alphaIFN), TNFalpha, an interleukin, such as IL-1 beta, IL-3, IL-4, IL-5, IL-S, IL-9, IL-13, IL 17 AF, IL-1S, IL13, IL-13R alphal, IL13R alpha2, IL14 IL-4R, IL-5R, IL-9R, IgE, blood group antigens, flk2/flt3 receptor, obesity (OB) receptor, mpl receptor, CTLA-4, RANKL, RANK, RSV F protein, protein C, BR3, etc.

In certain non-limiting embodiments, for example, the multispecific antibodies purified according to methods disclosed herein can target low density lipoprotein receptor-related protein (LRP)-1 or LRP-8 or transferrin receptor, and at least one target selected from the group consisting of 1) beta-secretase (BACE1 or BACE2), 2) alpha-secretase, 3) gamma-secretase, 4) tau-secretase, 5) amyloid precursor protein (APP), 6) death receptor 6 (DR6), 7) amyloid beta peptide, 8) alpha-synuclein, 9) Parkin, 10) Huntingtin, 11) p75 NTR, and 12) caspase-6.

In certain non-limiting embodiments, for example, the multispecific antibodies purified according to methods disclosed herein can target at least two target molecules selected from the group consisting of: IL-1 alpha and IL-1 beta, IL-12 and IL-1S, IL-13 and IL-9, IL-13 and IL-4, IL-13 and IL-5, IL-5 and IL-4, IL-13 and IL-1beta, IL-13 and IL-25, IL-13 and TARC, IL-13 and MDC, IL-13 and MEF, IL-13 and TGF, IL-13 and LHR agonist, IL-12 and TWEAK, IL-13 and CL25, IL-13 and SPRR2a, IL-13 and SPRR2b, IL-13 and ADAMS, IL-13 and PED2, IL13 and IL17, IL13 and IL4, IL13 and IL33, IL17A and IL 17F, CD3 and CD19, CD138 and CD20, CD138 and CD40, CD19 and CD20, CD20 and CD3, CD3S and CD13S, CD3S and CD20, CD3S and CD40, CD40 and CD20, CD-S and IL-6, CD20 and BR3, TNF alpha and TGF-beta, TNF alpha and IL-1 beta, TNF alpha and IL-2, TNF alpha and IL-3, TNF alpha and IL-4, TNF alpha and IL-5, TNF alpha and IL6, TNF alpha and IL8, TNF alpha and IL-9, TNF alpha and IL-10, TNF alpha and IL-11, TNF alpha and IL-12, TNF alpha and IL-13, TNF alpha and IL-14, TNF alpha and IL-15, TNF alpha and IL-16, TNF alpha and IL-17, TNF alpha and IL-18, TNF alpha and IL-19, TNF alpha and IL-20, TNF alpha and IL-23, TNF alpha and IFN alpha, TNF alpha and CD4, TNF alpha and VEGF, TNF alpha and MIF, TNF alpha and ICAM-1, TNF alpha and PGE4, TNF alpha and PEG2, TNF alpha and RANK ligand, TNF alpha and Te38, TNF alpha and BAFF, TNF alpha and CD22, TNF alpha and CTLA-4, TNF alpha and GP130, TNF a and IL-12p40, FGFR1 and KLB, VEGF and HER2, VEGF-A and HER2, VEGF-A and PDGF, HER1 and HER2, VEGFA and ANG2, VEGF-A and VEGF-C, VEGF-C and VEGF-D, HER2 and DRS, VEGF and IL-8, VEGF and MET, VEGFR and MET receptor, EGFR and MET, VEGFR and EGFR, HER2 and CD64, HER2 and CD3, HER2 and CD16, HER2 and HER3, EGFR (HER1) and HER2, EGFR and HER3, EGFR and HER4, IL-14 and IL-13, IL-13 and CD40L, IL4 and CD40L, TNFR1 and IL-1 R, TNFR1 and IL-6R and TNFR1 and IL-18R, EpCAM and CD3, MAPG and CD28, EGFR and CD64, CSPGs and RGM A, CTLA-4 and BTN02, IGF1 and IGF2, IGF1/2 and Erb2B, MAG and RGM A, NgR and RGM A, NogoA and RGM A, OMGp and RGM A, POL-1 and CTLA-4, and RGM A and RGM B.

Formulations and Methods of Making of the Formulations

The present disclosure provides formulations and methods of making the formulation comprising the multispecific antibodies purified by the methods described herein. For example, the purified polypeptide (e.g., the multispecific antibody) can be combined with a pharmaceutically acceptable carrier.

The polypeptide formulations in some embodiments may be prepared for storage by mixing a polypeptide having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some embodiments, the polypeptide in the polypeptide formulation maintains functional activity.

The formulations to be used for in vivo administration must be sterile. This is readily accomplished by filtration through sterile filtration membranes.

The formulations herein may also contain more than one active compound as necessary for the particular indication being treated, preferably those with complementary activities that do not adversely affect each other. For example, in addition to a polypeptide, it may be desirable to include in the one formulation, an additional polypeptide (e.g., antibody). Alternatively, or additionally, the composition may further comprise a chemotherapeutic agent, cytotoxic agent, cytokine, growth inhibitory agent, anti-hormonal agent, and/or cardioprotectant. Such molecules are suitably present in combination in amounts that are effective for the purpose intended

Articles of Manufacture

The present disclosure provides an article of manufacture comprising the multispecific antibodies purified by the methods described herein and/or formulations comprising the polypeptides purified by the methods described herein. The article of manufacture may comprise a container containing the polypeptide and/or the polypeptide formulation. In certain embodiments, the article of manufacture comprises: (a) a container comprising a composition comprising the polypeptide and/or the polypeptide formulation described herein within the container; and (b) a package insert with instructions for administering the formulation to a subject.

In certain embodiments, the article of manufacture comprises a container and a label or package insert on or associated with the container. Suitable containers include, for example, bottles, vials, syringes, etc. The containers may be formed from a variety of materials such as glass or plastic. The container holds or contains a formulation and may have a sterile access port (for example the container may be an intravenous solution bag or a vial having a stopper pierceable by a hypodermic injection needle). At least one active agent in the composition is the polypeptide. The label or package insert indicates that the composition's use in a subject with specific guidance regarding dosing amounts and intervals of polypeptide and any other drug being provided. The article of manufacture may further include other materials desirable from a commercial and user standpoint, including other buffers, diluents, filters, needles, and syringes. In some embodiments, the container is a syringe. In some embodiments, the syringe is further contained within an injection device. In some embodiments, the injection device is an autoinjector.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products.

Exemplary Embodiments of the Presently Disclosed Subject Matter

In certain embodiments, the present disclosure is directed to methods for purifying a multispecific antibody comprising: contacting a composition comprising the multispecific antibody and a mispaired variant thereof to a multi-mode chromatography material under conditions where the mispaired variant preferentially binds the multi-mode chromatographic material relative to the multispecific antibody, wherein the multispecific antibody comprises: a first antigen binding region specifically binding to a first antigen, wherein the first antigen binding region comprises the light chain and heavy chain of an antibody binding to the first antigen, and a second antigen binding region specifically binding to a second antigen, wherein the second antigen binding region comprises the light chain and heavy chain of an antibody binding to the second antigen, wherein in the second antigen binding region the variable domains VL and VH are replaced by each other; wherein the mispaired variant thereof comprises: a first antigen binding region comprising the heavy chain of the antibody binding to the first antigen and a peptide comprising the heavy chain variable domain (VH) and the light chain constant domain (CL) of the antibody binding to the second antigen, and a second antigen binding region comprising the light chain and heavy chain of an antibody binding to the second antigen, wherein in the second antigen binding region the variable domains VL and VH are replaced by each other; and wherein the multi-mode chromatography material comprises: a functional group capable of anion exchange, and a functional group capable of hydrophobic interactions; and collecting an eluate comprising the multispecific antibody and reduced amount of the mispaired variant thereof.

In certain embodiments of the methods described herein, the functional group capable of hydrophobic interactions comprises an alkyl-group, an alkenyl-group, an alkynyl-group, a phenyl-group, a benzyl-group, or any combination thereof.

In certain embodiments of the methods described herein, the functional group capable of hydrophobic interactions comprises a benzyl-group.

In certain embodiments of the methods described herein, the functional group capable of anion exchange comprises a positively charged group. In certain embodiments of the methods described herein, the positively charged group is a quaternary ammonium ion.

In certain embodiments of the methods described herein, the multi-mode chromatography material comprises a N-benzyl-N-methyl ethanolamine.

In certain embodiments of the methods described herein, the multi-mode chromatography material comprises a Capto™ Adhere resin.

In certain embodiments of the methods described herein, the multi-mode chromatography material comprises a Capto™ Adhere ImpRes resin.

In certain embodiments of the methods described herein, the elution of the multi-mode chromatography is a gradient elution. In certain embodiments of the methods described herein, the gradient elution comprises a pH gradient.

In certain embodiments of the methods described herein, the method comprises a capture chromatography step. In certain embodiments of the methods described herein, the capture chromatography step is an affinity chromatography step. In certain embodiments of the methods described herein, the affinity chromatography step is a protein A chromatography step, a protein L chromatography step, a protein G chromatography step, and a protein A/G chromatography step. In certain embodiments of the methods described herein, the affinity chromatography step is a protein A chromatography step. In certain embodiments of the methods described herein, the protein A chromatography step comprises a chromatographic material comprising protein A linked to agarose. In certain embodiments of the methods described herein, the capture chromatography step and the multi-mode chromatography step are contiguous. In certain embodiments of the methods described herein, the method comprises a purification step after the multi-mode chromatography step. In certain embodiments of the methods described herein, a concentration step where the multispecific antibody is concentrated.

In certain embodiments of the methods described herein, the multispecific antibody comprises a knob-in-hole modification.

In certain embodiments of the methods described herein, the multispecific antibody and the mispaired variant thereof are produced in the same host cell culture. In certain embodiments of the methods described herein, the host cell of the host cell culture is a prokaryotic cell or a eukaryotic cell. In certain embodiments of the methods described herein, the host cell is a eukaryotic cell. In certain embodiments of the methods described herein, the eukaryotic cell is a yeast cell, an insect cell, or a mammalian cell. In certain embodiments of the methods described herein, the eukaryotic cell is a CHO cell.

In certain embodiments, the present disclosure is directed to a composition comprising a multispecific antibody purified by the methods disclosed herein. In certain embodiments of the compositions described herein, the composition comprising a multispecific antibody comprises a pharmaceutically acceptable carrier.

In certain embodiments, the present disclosure relates to article of manufacture comprising a multispecific antibody purified by the methods disclosed herein.

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

The foregoing written description is considered to be sufficient to enable one skilled in the art to practice the methods and/or obtain the compositions described herein. The following examples and detailed description are offered by way of illustration and not by way of limitation.

The disclosures of all references in the specification are expressly incorporated herein by reference.

### EXAMPLES

The Examples are offered for illustrative purposes only, and are not intended to limit the scope of the present invention in any way. Indeed, various modifications in addition to those shown and described herein will become apparent to those skilled in the art from the foregoing description and fall within the scope of the appended claims.

It is understood that various other embodiments may be practiced, given the general description provided above.

### Example 1

One type of single-cell bispecific design is “CrossMab v2,” which improves light- and heavy-chain pairing by a design using Fab domain crossover. One of the possible light-chain (LC) mispairs places two variable-heavy (VH) domains in proximity. While it is generally understood that in antibodies VH domains only pair with variable-light (VL), the two VH domains in this LC-mispair may denature and produce structural distortions in the LC-mispaired Fab. Additionally, co-location of three negative-charge mutations on the heavy chain (HC, K147E, K213E) and LC (Q124E) may impart a negative charge patch on this LC-mispaired Fab.

The present example illustrate that a multi-mode chromatography resin (e.g., an anion exchange and hydrophobic-interaction chromatography (MMAEX)) can bind this LC-mispaired species and clear it in a downstream process, because the anion-exchange component interacts with the negatively-charged patch in the constant domain and the hydrophobic-interaction component binds to hydrophobic residues revealed by structural denaturation in the variable domain. Overall, this multi-mode chromatography improves the purification of multispecific antibodies.

Feedstock

An anti-Antigen A/anti-Antigen B bispecific antibody (aAgA/aAgB) was expressed as a CrossMab v2 with the domain crossover in the aAgB arm, in Chinese Hamster Ovary (CHO) cells. The resulting harvested cell culture fluid was purified by protein A affinity chromatography to capture the bispecific antibody and its product-related variants (e.g., unassembled half-antibodies, homodimers, and LC-mispair). The composition of the mixture was analyzed by reversed-phase HPLC and mass spectrometry and determined as described in the table below:

| % value | Multispecific Antibody Variant |
|---|---|
| 5.2% | aAgA knob half-antibody |
| 7.2% | aAgBhole half antibody |
| 0.8% | aAgA-aAgA knob-knob homodimer |
| <0.1% | LC-mispaired Bispecific (aAgA common LC) |
| 59.7% | Bispecific antibody (correctly formed, see FIGS. 2A |
| | and 2B) |
| 17.0% | LC-mispaired Bispecific (aAgB crossed LC, see FIGS. |
| | 2A and 2B) |
| 10.0% | aAgB-aAgB hole-hole homodimer |

High-Throughput Screening

An automated liquid-handling system was used to test binding of the feedstock to 5 different chromatography resins, including Capto Adhere (a MMAEX resin), under a variety of pH and buffer-strength conditions. Following incubation, the unbound fraction was analyzed and it was observed depletion of LC mispaired variant under conditions promoting anion-exchange behavior (high pH) and hydrophobic binding (high salt concentration) depicted in FIG. 3. Surprisingly, only the anion exchange and hydrophobic-interaction multi-mode chromatography resin was capable of binding this LC-mispaired species.

Confirmatory Column Chromatography Run

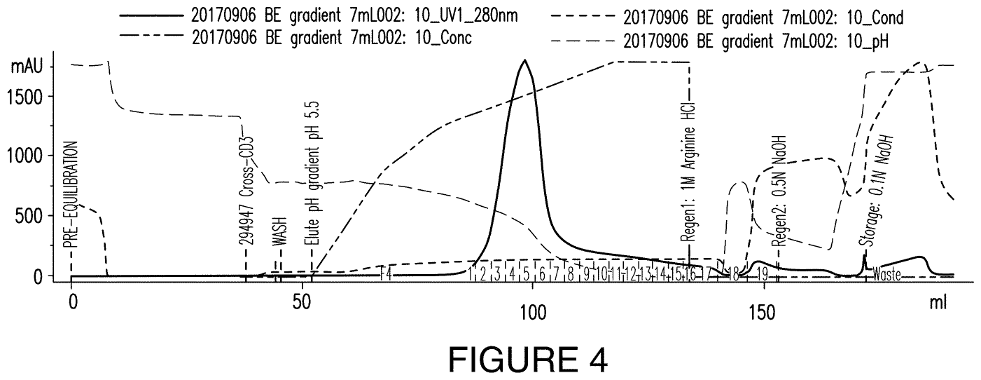

Using a Äkta chromatography system connected to a chromatography column with a packed-bed of Capto Adhere resin, a pH-adjusted feedstock was loaded onto the resin under strongly-binding conditions and then eluted using a pH-gradient from high pH (pH 8.6) to low pH (pH 5.5). Protein elution was observed as a main peak with a long tail (FIG. 4). The peak and tail were collected as fractions, which were then analyzed for composition.

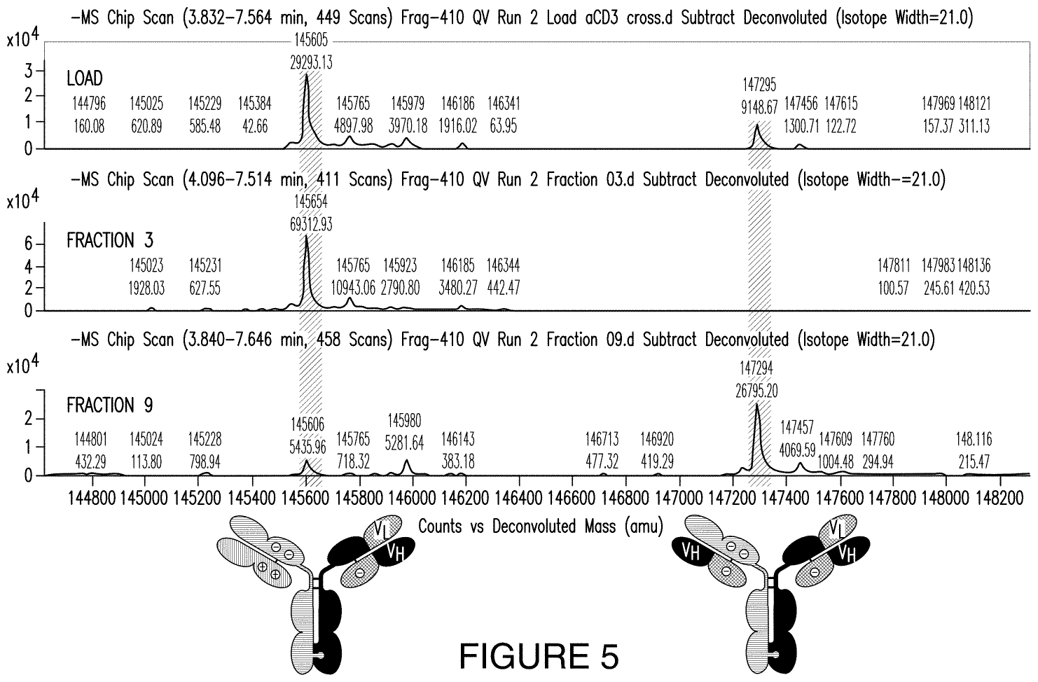

Analysis of the composition of collected fractions revealed that the main elution peak was enriched in bispecific antibody, while the post-peak tail was enriched in LC-mispair. FIG. 5 shows mass spectra comparing load feedstock composition (LOAD) to a fraction representing the main peak enriched with bispecific (FRACTION 3) and a fraction representing the post-peak tail enriched with LC mispair (FRACTION 9)).

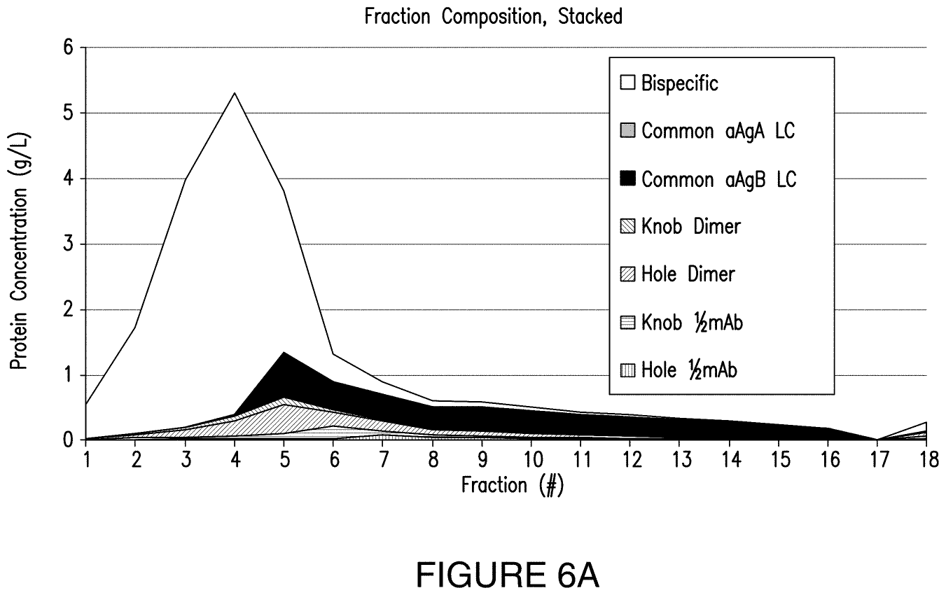

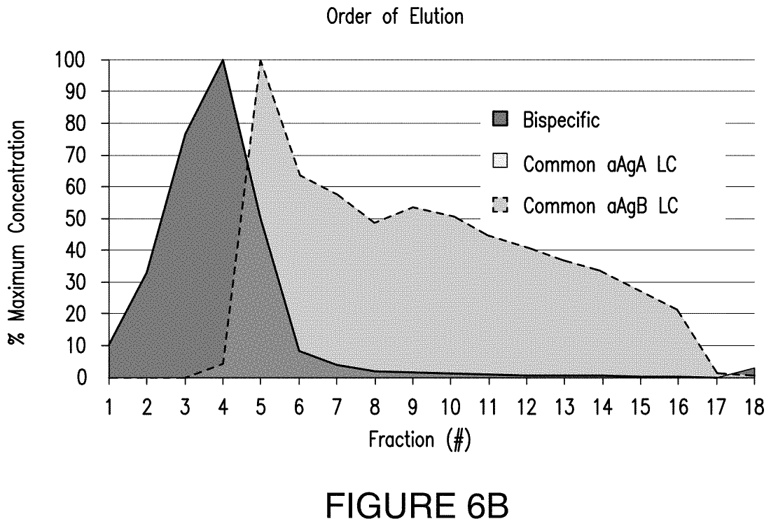

To further evaluate the role of the methods disclosed herein, pseudo-chromatograms depicting composition and concentration of collected and measured fractions were analyzed. As illustrated in FIG. 6A, the main peak comprised primarily bispecific antibody, while the post-peak tail comprised primarily LC-mispair variant. Other product-related variants were present in minor levels. When the pseudo-chromatograms for bispecific and LC-mispair were normalized (e.g., scaled to same height) and overlaid, it was more apparent that the method disclosed herein separated bispecific antibody from LC mispair variants (FIG. 6B).

Molecular Structure Study

Next, in support of the experimental findings, 3D homology models of this molecules' Fabs with both correctly and incorrectly-paired HC and LC combinations were prepared. It was observed that the LC-mispaired Fab did exhibit a loose and denatured variable-domain structure (as VH domains are understood not to have affinity for other VH domains). Furthermore, the three negatively-charged amino acids are located on the surface of the protein, and therefore able to create a patch of negative charge on the surface of the protein at the constant domain.

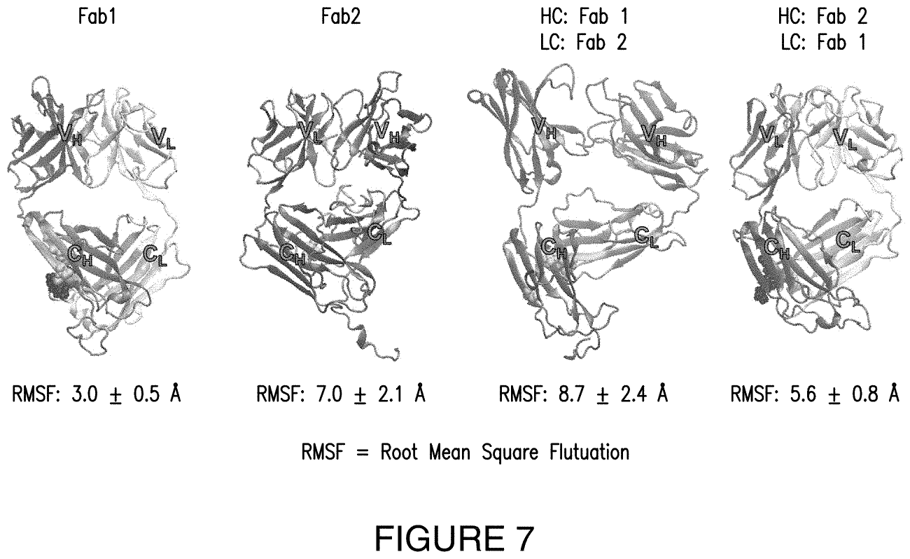

Simulated structures of correct paired (FIGS. 7A and 7B) and LC-mispaired (FIGS. 7C and 7D) species. The LC-mispaired species exhibiting highest structural distortion as well as a negative-charge cluster were removed (FIG. 7C).

### CONCLUSION

Some degree of LC mispairing is unavoidable in single-cell bispecific designs. LC-mispairs are extremely difficult to remove from the correctly-formed bispecific, however if a particular combination of LC and HC results in a product-related variant that is suspected to present risk (for example, risk to a patient), then the single-cell bispecific can be designed in a way that improves the ability to remove a particular LC mispair. The method disclosed herein can remove a LC-mispair from a Crossmab v2 bispecific, as long as the mispair is between a crossed-LC and an uncrossed HC.

What is claimed is:

1. A method for purifying a multispecific antibody, comprising:
a) contacting a composition comprising the multispecific antibody and a mispaired variant thereof to a multi-mode chromatography material under conditions where the mispaired variant preferentially binds the multi-mode chromatographic material relative to the multispecific antibody,
i) wherein the multispecific antibody comprises:
1) a first antigen binding region specifically binding to a first antigen, wherein the first antigen binding region comprises the light chain and heavy chain of an antibody binding to the first antigen, and
2) a second antigen binding region specifically binding to a second antigen, wherein the second antigen binding region comprises the light chain and heavy chain of an antibody binding to the second antigen, wherein in the second antigen binding region the variable domains VL and VH are replaced by each other;
ii) wherein the mispaired variant thereof comprises:
1) a first antigen binding region comprising the heavy chain of the antibody binding to the first antigen and a peptide comprising the heavy chain variable domain (VH) and the light chain constant domain (CL) of the antibody binding to the second antigen, and
2) a second antigen binding region comprising the light chain and heavy chain of an antibody binding to the second antigen, wherein in the second antigen binding region the variable domains VL and VH are replaced by each other; and
iii) wherein the multi-mode chromatography material comprises:
1) a functional group capable of anion exchange, and
2) a functional group capable of hydrophobic interactions; and
b) collecting an eluate comprising the multispecific antibody and reduced amount of the mispaired variant thereof.

2. The method of claim 1, wherein the functional group capable of hydrophobic interactions comprises an alkyl-group, an alkenyl-group, an alkynyl-group, a phenyl-group, a benzyl-group, or any combination thereof.

3. The method of claim 2, wherein the functional group capable of hydrophobic interactions comprises a benzyl-group.

4. The method of any one of claims 1-3, wherein the functional group capable of anion exchange comprises a positively charged group.

5. The method of claim 4, wherein the positively charged group is a quaternary ammonium ion.

6. The method of any one of claims 1-5, wherein the multi-mode chromatography material comprises a N-benzyl-N-methyl ethanolamine.

7. The method of any one of claims 1-6, wherein the multi-mode chromatography material comprises a Capto™ Adhere resin.

8. The method of any one of claims 1-6, wherein the multi-mode chromatography material comprises a Capto™ Adhere ImpRes resin.

9. The method of any one of claims 1-8, wherein the elution of the multi-mode chromatography is a gradient elution.

10. The method of claim 9, wherein the gradient elution comprises a pH gradient.

11. The method of any one of claims 1-10, wherein the method comprises a capture chromatography step.

12. The method of claim 11, wherein the capture chromatography step is an affinity chromatography step.

13. The method of claim 12, wherein the affinity chromatography step is a protein A chromatography step, a protein L chromatography step, a protein G chromatography step, and a protein A/G chromatography step.

14. The method of claim 12 or 13, wherein the affinity chromatography step is a protein A chromatography step.

15. The method of claim 14, wherein the protein A chromatography step comprises a chromatographic material comprising protein A linked to agarose.

16. The method of any one of claims 11-15, wherein the capture chromatography step and the multi-mode chromatography step are contiguous.

17. The method of any one of claims 1-16, wherein the method comprises a purification step after the multi-mode chromatography step.

18. The method of any one of claims 1-17, comprising a concentration of the multispecific antibody.

19. The method of any one of claims 1-18, wherein the multispecific antibody comprises a knob-in-hole modification.

20. The method of any one of claims 1-19, wherein the multispecific antibody and the mispaired variant thereof are produced in the same host cell culture.

21. The method of claim 20, wherein the host cell of the host cell culture is a prokaryotic cell or a eukaryotic cell.

22. The method of claim 20 or 21, wherein the host cell is a eukaryotic cell.

23. The method of claim 22, wherein the eukaryotic cell is a yeast cell, an insect cell, or a mammalian cell.

24. The method of claim 22 or 23, wherein the eukaryotic cell is a CHO cell.

25. A composition comprising a multispecific antibody purified by the method of any one of claims 1-24.

26. The composition of claim 25 comprising a pharmaceutically acceptable carrier.

27. An article of manufacture comprising a multispecific antibody purified by the method of any one of claims 1-24 or a composition of claim 25 or 26.
